SEQUENCE LISTING (67 sequences)

SEQ ID NO: 1 - DNA, 66 bp, Artificial Sequence
  miRNA-668
ggtaagtgcgcctcgggtgagcatgcacttaatgtgggtgtatgtcactcggctcggcccactacc

SEQ ID NO: 2 - DNA, 66 bp, Artificial Sequence
  miRNA-376c
aaaaggtggatattccttctatgtttatgttatttatggttaaacatagaggaaattccacgtttt

SEQ ID NO: 3 - DNA, 68 bp, Artificial Sequence
  miRNA-376a
taaaaggtagattctccttctatgagtacattatttatgattaatcatagaggaaaatccacgttttc

SEQ ID NO: 4 - DNA, 81 bp, Artificial Sequence
  miRNA-494
gatactcgaaggagaggttgtccgtgttgtcttctctttatttatgatgaaacatacacgggaaacctcttttttagtatc

SEQ ID NO: 5 - DNA, 84 bp, Artificial Sequence
  miRNA-541
acgtcagggaaaggattctgctgtcggtcccactccaaagttcacagaatgggtggtgggcacagaatctggactctgcttgtg

SEQ ID NO: 6 - DNA, 88 bp, Artificial Sequence
  miRNA-1197
acttcctggtatttgaagatgcggttgaccatggtgtgtacgctttatttgtgacgtaggacacatggtctacttcttctcaatatca

SEQ ID NO: 7 - DNA, 82 bp, Artificial Sequence
  miRNA-495
tggtacctgaaaagaagttgcccatgttattttcgctttatatgtgacgaaacaaacatggtgcacttctttttcggtatca

SEQ ID NO: 8 - DNA, 76 bp, Artificial Sequence
  miRNA-382
tacttgaagagaagttgttcgtggtggattcgctttacttatgacgaatcattcacggacaacacttttttcagta

SEQ ID NO: 9 - DNA, 91 bp, Artificial Sequence
  miRNA-412
ctggggtacggggatggatggtcgaccagttggaaagtaattgtttctaatgtacttcacctggtccactagccgtccgtatccgctgcag

SEQ ID NO: 10 - DNA, 69 bp, Artificial Sequence
  miRNA-377
ttgagcagaggttgcccttggtgaattcgctttatttatgttgaatcacacaaaggcaacttttgtttg

SEQ ID NO: 11 - DNA, 80 bp, Artificial Sequence
  miRNA-329
ggtacctgaagagaggttttctgggtttctgtttctttaatgaggacgaaacacacctggttaacctcttttccagtatc

SEQ ID NO: 12 - DNA, 88 bp, Artificial Sequence
  miRNA-758
gcctggatacatgagatggttgaccagagagcacacgctttatttgtgccgtttgtgacctggtccactaaccctcagtatctaatgc

SEQ ID NO: 13 - DNA, 61 bp, Artificial Sequence
  miRNA-380
aagatggttgaccatagaacatgcgctatctctgtgtcgtatgtaatatggtccacatctt

SEQ ID NO: 14 - DNA, 83 bp, Artificial Sequence
  miRNA-300
tgctacttgaagagaggtaatccttcacgcatttgctttacttgcaatgattatacaagggcagactctctctggggagcaaa

SEQ ID NO: 15 - DNA, 63 bp, Artificial Sequence
  miRNA-299
aagaaatggtttaccgtcccacatacattttgaatatgtatgtgggatggtaaaccgcttctt

SEQ ID NO: 16 - DNA, 79 bp, Artificial Sequence
  miRNA-409
tggtactcggggagaggttacccgagcaactttgcatctggacgacgaatgttgctcggtgaaccccttttcggtatca

SEQ ID NO: 17 - DNA, 73 bp, Artificial Sequence
  miRNA-134
cagggtgtgtgactggttgaccagaggggcatgcactgtgttcaccctgtgggccacctagtcaccaaccctc

SEQ ID NO: 18 - DNA, 91 bp, Artificial Sequence
  miRNA-544a
attttcatcacctagggatcttgttaaaaagcagattctgattcagggaccaagattctgcatttttagcaagttctcaagtgatgctaat

SEQ ID NO: 19 - DNA, 67 bp, Artificial Sequence
  miRNA-379
agagatggtagactatggaacgtaggcgttatgatttctgacctatgtaacatggtccactaactct

SEQ ID NO: 20 - DNA, 94 bp, Artificial Sequence
  miRNA-432
tgactcctccaggtcttggagtaggtcattgggtggatcctctatttccttacgtgggccactggatggctcctccatgtcttggagtagatca

SEQ ID NO: 21 - DNA, 89 bp, Artificial Sequence
  miRNA-493
ctggcctccagggctttgtacatggtaggctttcattcattcgtttgcacattcggtgaaggtctactgtgtgccaggccctgtgccag

SEQ ID NO: 22 - DNA, 82 bp, Artificial Sequence
  miRNA-136
tgagccctcggaggactccatttgttttgatgatggattcttatgctccatcatcgtctcaaatgagtcttcagagggttct

SEQ ID NO: 23 - DNA, 114 bp, Artificial Sequence
  miRNA-431
tcctgcttgtcctgcgaggtgtcttgcaggccgtcatgcaggccacactgacggtaacgttgcaggtcgtcttgcagggcttctcgcaagacgacatcctcatcaccaacgacg

SEQ ID NO: 24 - DNA, 84 bp, Artificial Sequence
  miRNA-487b
ttggtacttggagagtggttatccctgtcctgttcgttttgctcatgtcgaatcgtacagggtcatccactttttcagtatcaa

SEQ ID NO: 25 - DNA, 100 bp, Artificial Sequence
  miRNA-376b
cagtccttctttggtatttaaaacgtggatattccttctatgtttacgtgattcctggttaatcatagaggaaaatccatgttttcagtatcaaatgctg

SEQ ID NO: 26 - DNA, 72 bp, Artificial Sequence
  miRNA-665
tctcctcgaggggtctctgcctctacccaggactctttcatgaccaggaggctgaggcccctcacaggcggc

SEQ ID NO: 27 - DNA, 81 bp, Artificial Sequence
  miRNA-654
gggtaagtggaaagatggtgggccgcagaacatgtgctgagttcgtgccatatgtctgctgaccatcacctttagaagccc

SEQ ID NO: 28 - DNA, 75 bp, Artificial Sequence
  miRNA-370
agacagagaagccaggtcacgtctctgcagttacacagctcacgagtgcctgctggggtggaacctggtctgtct

SEQ ID NO: 29 - DNA, 86 bp, Artificial Sequence
  miRNA-323a
ttggtacttggagagaggtggtccgtggcgcgttcgctttatttatggcgcacattacacggtcgacctctttgcagtatctaatc

SEQ ID NO: 30 - DNA, 78 bp, Artificial Sequence
  miRNA-543
tacttaatgagaagttgcccgtgtttttttcgctttatttgtgacgaaacattcgcggtgcacttctttttcagtatc

SEQ ID NO: 31 - DNA, 102 bp, Artificial Sequence
  miRNA-496
cccaagtcaggtactcgaatggaggttgtccatggtgtgttcattttatttatgatgagtattacatggccaatctcctttcggtactcaattcttcttggg

SEQ ID NO: 32 - DNA, 78 bp, Artificial Sequence
  miRNA-539
atacttgaggagaaattatccttggtgtgttcgctttatttatgatgaatcatacaaggacaatttctttttgagtat

SEQ ID NO: 33 - DNA, 75 bp, Artificial Sequence
  miRNA-381
tacttaaagcgaggttgccctttgtatattcggtttattgacatggaatatacaagggcaagctctctgtgagta

SEQ ID NO: 34 - DNA, 84 bp, Artificial Sequence
  miRNA-154
gtggtacttgaagataggttatccgtgttgccttcgctttatttgtgacgaatcatacacggttgacctatttttcagtaccaa

SEQ ID NO: 35 - DNA, 78 bp, Artificial Sequence
  miRNA-1193
gtagctgaggggatggtagaccggtgacgtgcacttcatttacgatgtaggtcacccgtttgactatccaccagcgcc

SEQ ID NO: 36 - DNA, 93 bp, Artificial Sequence
  miRNA-337
gtagtcagtagttggggggtgggaacggcttcatacaggagttgatgcacagttatccagctcctatatgatgcctttcttcatccccttcaa

SEQ ID NO: 37 - DNA, 70 bp, Artificial Sequence
  miRNA-369
ttgaagggagatcgaccgtgttatattcgctttattgacttcgaataatacatggttgatcttttctcag

SEQ ID NO: 38 - DNA, 93 bp, Artificial Sequence
  miRNA-433
ccggggagaagtacggtgagcctgtcattattcagagaggctagatcctctgtgttgagaaggatcatgatgggctcctcggtgttctccagg

SEQ ID NO: 39 - DNA, 94 bp, Artificial Sequence
  miRNA-434
tcgactctgggtttgaaccaaagctcgactcatggtttgaaccattacttaattcgtggtttgaaccatcactcgactcctggttcgaaccatc

SEQ ID NO: 40 - DNA, 73 bp, Artificial Sequence
  miRNA-485
acttggagagaggctggccgtgatgaattcgattcatcaaagcgagtcatacacggctctcctctcttttagt

SEQ ID NO: 41 - DNA, 80 bp, Artificial Sequence
  miRNA-410
ggtacctgagaagaggttgtctgtgatgagttcgcttttattaatgacgaatataacacagatggcctgttttcagtacc

SEQ ID NO: 42 - DNA, 96 bp, Artificial Sequence
  miRNA-411
tggtacttggagagatagtagaccgtatagcgtacgctttatctgtgacgtatgtaacacggtccactaaccctcagtatcaaatccatccccgag

SEQ ID NO: 43 - DNA, 97 bp, Artificial Sequence
  miRNA-127
tgtgatcactgtctccagcctgctgaagctcagagggctctgattcagaaagatcatcggatccgtctgagcttggctggtcggaagtctcatcatc

SEQ ID NO: 44 - DNA, 22 bp, Artificial Sequence
  mouse atrogin-1 forward primer
acaagggaagtacgaaggagcg

SEQ ID NO: 45 - DNA, 20 bp, Artificial Sequence
  mouse atrogin-1 reverse primer
ggcagtcgagaagtccagtc

SEQ ID NO: 46 - DNA, 18 bp, Artificial Sequence
  mouse beta-actin forward primer
ggctgtattcccctccat

SEQ ID NO: 47 - DNA, 21 bp, Artificial Sequence
  mouse beta-actin reverse primer
ccagttggtaacaatgccatg

SEQ ID NO: 48 - DNA, 19 bp, Artificial Sequence
  human atrogin-1 forward primer
ccatccgtctagtccgctc

SEQ ID NO: 49 - DNA, 20 bp, Artificial Sequence
  human atrogin-1 reverse primer
tgaggtcgctcacgaaactg

SEQ ID NO: 50 - DNA, 18 bp, Artificial Sequence
  human GAPDH forward primer
tgttgccatcaatgaccc

SEQ ID NO: 51 - DNA, 18 bp, Artificial Sequence
  human GAPDH reverse primer
cccacgacgtactcagcg

SEQ ID NO: 52 - DNA, 21 bp, Artificial Sequence
  mmu-miR-376c primer
aacatagaggaaatttcacgt

SEQ ID NO: 53 - DNA, 19 bp, Artificial Sequence
  U6 primer
tggcccctgcgcaaggatg

SEQ ID NO: 54 - DNA, 19 bp, Artificial Sequence
  atrogin-1 forward primer
cagcttcgtgagcgacctc

SEQ ID NO: 55 - DNA, 20 bp, Artificial Sequence
  atrogin-1 reverse primer
ggcagtcgagaagtccagtc

SEQ ID NO: 56 - DNA, 20 bp, Artificial Sequence
  GAPDH forward primer
gggaaattcaacggcacagt

SEQ ID NO: 57 - DNA, 19 bp, Artificial Sequence
  GAPDH reverse primer
agatggtgatgggcttccc

SEQ ID NO: 58 - DNA, 20 bp, Artificial Sequence
  Luciferase 2 forward primer
caccttcgtgacttcccatt

SEQ ID NO: 59 - DNA, 20 bp, Artificial Sequence
  Luciferase 2 reverse primer
tgactgaatcggacacaagc

SEQ ID NO: 60 - DNA, 20 bp, Artificial Sequence
  5′biotinylated ASO for endogenous Atrogin-1 primer
atgtggcactcacagcagag

SEQ ID NO: 61 - DNA, 20 bp, Artificial Sequence
  5′biotinylated ASO for reporter mRNA primer
agacgggcaagaaagaggat

SEQ ID NO: 62 - RNA, 19 nt, Artificial Sequence
  Atrogin-1 Cat. No. 1357211 sense sequence
gauagauguguucgucuua

SEQ ID NO: 63 - RNA, 19 nt, Artificial Sequence
  Atrogin-1 Cat. No. 1357211 antisense sequence
uaagacgaacacaucuauc

SEQ ID NO: 64 - RNA, 19 nt, Artificial Sequence
  Atrogin-1 Cat. No. 1357212 sense sequence
gugaucuaagaugggaagg

SEQ ID NO: 65 - RNA, 19 nt, Artificial Sequence
  Atrogin-1 Cat. No. 1357212 antisense sequence
ccuucccaucuuagaucac

SEQ ID NO: 66 - RNA, 19 nt, Artificial Sequence
  Atrogin-1 Cat. No. 1357210 sense sequence
agagagucggcaagucugu

SEQ ID NO: 67 - RNA, 19 nt, Artificial Sequence
  Atrogin-1 Cat. No. 1357210 antisense sequence
acagacuugccgacucucu

(19) United States
(12) Patent Application Publication
(10) Pub. No.: US 2021/0261969 A1
(43) Pub. Date: Aug. 26, 2021

(54) PHARMACEUTICAL COMPOSITION FOR PREVENTING OR TREATING MUSCULAR DISEASE OR CACHEXIA COMPRISING, AS ACTIVE INGREDIENT, miRNA LOCATED IN DLK1-DIO3 CLUSTER OR VARIANT THEREOF

(71) Applicant: KOREA RESEARCH INSTTITUTE OF BIOSCIENAND BIOTECHNOLOGY, Daejeon (KR)

(72) Inventors: Ki-Sun KWON, Daejeon (KR); Kwang-Pyo LEE, Daejeon (KR); Yeo Jin SHIN, Daejeon (KR); Bora LEE, Daejeon (KR); Seung Min LEE, Daejeon (KR)

(21) Appl. No.: 16/961,130

(22) PCT Filed: Jan. 9, 2019
(86) PCT No.: PCT/KR2019/000344

§ 371 Date: Jul. 9, 2020

(30) Foreign Application Priority Data
Jan. 9, 2018 (KR) 10-2018-0002557

(51) Int. Cl. C12N 15/113; A61P 21/00

(52) U.S. Cl. CPC C12N 15/1137; C12N 2310/141; A61P 21/00

(57) ABSTRACT

The present invention relates to a pharmaceutical composition for preventing or treating a muscular disease or cachexia, comprising, as an active ingredient, a miRNA located in Dlk1-Dio3 cluster or a variant thereof. In the present invention, it has been found that expression of miRNAs located in the Dlk1-Dio3 cluster is decreased as age increases. In particular, in a case where most of the miRNAs are over-expressed in fully differentiated myotubes, it has been confirmed that the diameter of the myotubes increases. In addition, also in a tumor-induced cachexia mouse model, it has been confirmed that cachexia was improved by inhibiting Atrogin-1 protein. Accordingly, the miRNA located in the Dlk1-Dio3 cluster or a variant thereof can be usefully utilized for the treatment and prevention of an Atrogin-1-dependent muscular disease and cachexia.

### TECHNICAL FIELD

The present invention relates to a pharmaceutical composition for preventing or treating a muscular disease or cachexia, comprising, as an active ingredient, a miRNA located in Dlk1-Dio3 cluster or a variant thereof.

### BACKGROUND ART

Mass and function of skeletal muscles gradually decrease with age, which is a major cause of mortality and poor quality of life for the elderly. Aged skeletal muscles show not only decrease in muscle mass but also progressive decrease in strength and function. This disease is called “aging-induced sarcopenia” (Jun-Won Heo and et al., Aging-induced Sarcopenia and Exercise. The Official Journal of the Korean Academy of Kinesiology, 19(2). DOI: http://doi.org/10.15758/jkak.2017.19.2.43). Muscle mass decreases by about 1% every year in the 30s. Prevalence of aging-induced sarcopenia is about 10% in the elderly in their 60s and increases to about 50% in their 80s. Decrease of muscle with aging triggers disability in physical activity as well as various diseases such as type 2 diabetes, obesity, dyslipidemia, and hypertension. Thus, it is urgent to develop an effective therapeutic agent for healthy muscles. However, to date, there is no therapeutic agent for aging-induced sarcopenia which has been approved by the Food and Drug Administration (FDA). Recently, for aging-induced sarcopenia, the World Health Organization has assigned a disease code thereof to the International Classification of Diseases, 10th Revision, Clinical Modification (ICD-10-CM). Given this situation, it is expected that development of diagnostic and therapeutic agents for aging-induced sarcopenia will be accelerated.

Muscle mass is determined by dynamic balance between anabolism and catabolism. Muscular atrophy has been reported to occur through various stimuli including interleukin-1 (IL-1), tumor necrosis factor (TNF-α), and glucocorticoid. In such muscular atrophy, it is known that muscle-specific E3 ligases (for example, MuRF1 and Atrogin-1/MAFbx) play an important role. Such E3 ligases have been reported to remarkably increase in various diseases such as nerve damage, diabetes, sepsis, hyperthyroidism, and cancer-induced cachexia in a case where muscles are not moved for a long time. Little is known about an E3 ligase regulation mechanism in aged muscles, and only gene expression levels of MuRF1 and Atrogin-1 in mouse, rat and human muscles are known. However, these study results for gene expression studies are also controversial in view of the conflicting results offered by another study group, and the like. Meanwhile, a microRNA (hereinafter referred to as miRNA) is one of the most widely studied non-coding RNAs, and has a main role to regulate expression of a gene at post-transcriptional level. The microRNAs, each of which is a single-stranded molecule consisting of about 22 nucleotides, are often disposed in a polycistronic cluster and tend to jointly target the same target or the same pathway. Delta-like 1 homolog-type 3 iodothyronine deiodinase (Dlk1-Dio3) is the largest known miRNA cluster. Little is known about functions of the Dlk1-Dio3 cluster in muscle aging.

### DISCLOSURE

### Technical Problem

Accordingly, the present inventors intended to examine whether miRNAs located in the Dlk1-Dio3 cluster play any common role in decrease of muscle caused by aging, and, based on the examination, to confirm a possibility of their use as a therapeutic agent for aging-induced sarcopenia. As a result, the present inventors confirmed that the miRNAs located in the Dlk1-Dio3 cluster are involved in aging of skeletal muscles and myoblasts of mice. In addition, the present inventors elucidated a miRNA-mediated Atrogin-1 expression regulation mechanism in a muscle aging process, and confirmed that a genetic therapeutic method based on the miRNAs in the Dlk1-Dio3 cluster has effective prophylactic efficacy on muscle aging as well as cancer-induced cachexia, thereby completing the present invention.

An object of the present invention is to provide a composition for preventing or treating muscle aging or cachexia, comprising, as an active ingredient, a miRNA in Dlk1-Dio3 cluster.

### Technical Solution

In order to achieve the above object, the present invention provides a pharmaceutical composition for preventing or treating a muscular disease, comprising, as an active ingredient, a miRNA located in Dlk1-Dio3 cluster or a variant thereof.

In addition, the present invention provides a pharmaceutical composition for preventing or treating a muscular disease, comprising, as an active ingredient, a vector loaded with a nucleotide encoding a miRNA located in Dlk1-Dio3 cluster or a variant thereof

In addition, the present invention provides a pharmaceutical composition for preventing or treating cachexia, comprising, as an active ingredient, a miRNA located in Dlk1-Dio3 cluster or a variant thereof.

In addition, the present invention provides a pharmaceutical composition for preventing or treating cachexia, comprising, as an active ingredient, a vector loaded with a nucleotide encoding a miRNA located in Dlk1-Dio3 cluster or a variant thereof.

In addition, the present invention provides a method for preventing or treating a muscular disease, comprising a step of administering to a subject a pharmaceutical composition for preventing or treating the muscular disease.

In addition, the present invention provides a method for preventing or treating cachexia, comprising a step of administering to a subject a pharmaceutical composition for preventing or treating cachexia.

In addition, the present invention provides a use of a miRNA located in Dlk1-Dio3 cluster or a variant thereof for the manufacture of a medicament for preventing or treating a muscular disease.

In addition, the present invention provides a use of a vector loaded with a nucleotide encoding a miRNA located in Dlk1-Dio3 cluster or a variant thereof for the manufacture of a medicament for preventing or treating a muscular disease.

In addition, the present invention provides a use of a miRNA located in Dlk1-Dio3 cluster or a variant thereof for the manufacture of a medicament for preventing or treating cachexia.

In addition, the present invention provides a use of a vector loaded with a nucleotide encoding a miRNA located in Dlk1-Dio3 cluster or a variant thereof for the manufacture of a medicament for preventing or treating cachexia.

### Advantageous Effects

In the present invention, it has been found that expression of miRNAs located in the Dlk1-Dio3 cluster decreases with aging. In addition, it has been confirmed that in a case where specific miRNAs are over-expressed in fully differentiated myotubes, the diameter of the myotubes increases. In addition, it has been confirmed that various miRNAs in the Dlk1-Dio3 cluster interact with Atrogin-1 3′-UTR so that protein expression of Atrogin-1, which is a muscle-specific E3 ligase, is suppressed. In addition, in a case where muscles of aged mice are infected with adenovirus expressing the miRNA, skeletal muscular atrophy was dramatically improved. In addition, it has been confirmed that even in a tumor-induced cachexia mouse model, cachexia was improved by inhibiting Atrogin-1 protein using the miRNA. Therefore, the miRNA located in the Dlk1-Dio3 cluster or a variant thereof can be usefully utilized to prevent an Atrogin-1-dependent muscular disease and to improve cachexia.

### DESCRIPTION OF DRAWINGS

FIGS. 1a to 1d illustrate results obtained by making a comparative analysis between miRNA expression profiles for tibialis anterior (TA) muscles and muscle fibers isolated from young mice and old mice.

Specifically, FIG. 1a illustrates a chart indicating distribution of miRNAs which are differentially expressed in TA muscle tissue by age. The pie chart on the left shows that 56% (61) of the miRNAs in aged TA muscles exhibit decreased expression. The doughnut chart on the right shows that 69% (42) of the miRNAs with decreased expression are located in a Dlk-Dio3 genome region.

FIG. 1b illustrates classification with aging, depending on an expression level (>1.5-fold), of 109 miRNAs in which 48 miRNAs with increased expression and 61 miRNAs with decreased expression are used.

FIG. 1c illustrates classification with aging, depending on an expression level, of 42 miRNAs located in the Dlk1-Dio3 genome region. The respective columns show miRNA expression levels in TA muscles of young mice (6 months) and old mice (24 months).

FIG. 1d illustrates a chart showing distribution of miRNAs which are differentially expressed in myoblasts isolated from young mice (3 months) and old mice (27 months). The pie chart on the left shows that 60% (71) of the miRNAs in aged myoblasts exhibit decreased expression. The doughnut chart on the right shows that 83% (59) of the miRNAs with decreased expression are located in the Dlk-Dio3 genome region.

FIG. 1e illustrates classification, depending on an expression level (>2-fold), of 118 miRNAs in which 47 miRNAs with increased expression and 71 miRNAs with decreased expression are used.

FIG. 1f illustrates classification with aging, depending on an expression level, of 59 miRNAs located in the Dlk1-Dio3 genome region. The respective rows show miRNA levels in myoblasts isolated from young or old TA muscles.

FIG. 2 illustrates changes, with aging, in expression level of miRNAs present in the Dlk1-Dio3 cluster by age. Specifically, correlation between a human age (25 to 80 years old) and an expression level of 18 miRNAs present in human Dlk1-Dio3 cluster is illustrated. The miRNAs were isolated from human gluteus maximus muscles. Data were evaluated using Spearman's correlation test (ρ; 95% CI; n=20).

FIG. 3a illustrates a screening plan for miRNAs that lead to a muscle hypertrophy phenotype. On day 4 after induction of differentiation of C2C12 cells, miRNA mimics were individually transfected into differentiated myotubes to confirm activity of miRNAs present in the Dlk1-Dio3 cluster. Diameters of the myotubes were measured 24 hours after transfection.

FIG. 3b illustrates images of differentiated muscle cells transfected with miRNA mimics. Myotubes were stained with Eosin Y for diameter measurement. A scale bar is 50 μm.

FIG. 3c illustrates percentages of myotubes having various diameters after transfection with miRNA mimics. A darker color indicates a larger diameter. Four images were randomly selected for diameter measurement using a microscope imaging software (NIS-Elements Basic Research, Nikon). Data were presented as mean ±SD.

FIG. 4a illustrates that in mouse Atrogin-1 3′UTR, 38 binding sites for miRNAs (among which 12 are conserved in humans) are predicted.

FIG. 4b illustrates relative activity of a luciferase reporter having Atrogin-1 3′UTR in 293T cells transfected with miRNA (*P<0.05, **P<0.01, ***P<0.001).

FIG. 4c illustrates immunoblot assay results in differentiated C2C12 cells transfected with miRNA. The results were normalized with GAPDH.

FIG. 4d illustrates quantification of relative expression levels of Atrogin-1 in FIG. 4c.

FIG. 4e illustrates immunoblot assay results for Atrogin-1 in C2C12 myotubes transfected with miR-493, miR-376b, and miR-433. Expression levels of Atrogin-1 were quantified using ImageJ software and the results were normalized with GAPDH expression.

FIG. 4f illustrates immunoblot assay results of differentiated human skeletal muscle myoblasts (HSMMs) transfected with miRNA. The results were normalized with GAPDH.

FIG. 4g illustrates quantification of relative expression levels of Atrogin-1 in FIG. 4f.

FIGS. 4h and 4i illustrate immunoassay and quantification results for Atrogin-1 in TA muscles isolated from young mice or old mice. The results were normalized with a mean expression level of ACTN1 (***P<0.001).

FIG. 4j illustrates immunoblot assay results for Atrogin-1 in human muscle tissue at indicated ages. The results were normalized with GAPDH.

FIG. 4k illustrates results of correlation analysis between a human age and expression of Atrogin-1. The results were evaluated using Spearman's correlation test (p; 95% CI).

FIG. 4l illustrates relative expression levels of Atrogin-1 mRNA in TA muscles isolated from young mice or old mice. The results were normalized with ACTB.

FIG. 4m illustrates expression levels of Atrogin-1 mRNA in muscles of human subjects aged from 25 and 80. The results were normalized with GAPDH. The results were evaluated using Spearman's correlation test (p; 95% CI; n=20).

FIG. 4n illustrates miRNAs included in stepwise analysis. Here, * is conserved in humans.

FIG. 5a illustrates proportions of muscle weight and body weight in old mice.

FIG. 5b illustrates cross-sectional images of muscles in young mice and old mice (red, laminin; blue, DAPI; scale bar, 50 μm).

FIG. 5c illustrates morphological analysis results for cross section area (CSA). Four different images were randomly selected and each CSA was analyzed using ImageJ software (*P<0.05).

FIG. 6 illustrates expression of top 5 miRNAs that strongly lead to a C2C12 muscle hypertrophy phenotype in young TA muscles and old TA muscles. Specifically, relative expression levels of miR-668, miR-376c, miR-494, miR-541, and miR-1197 in young or old TA muscles (n=5) are illustrated. The results were normalized to U6 small nuclear RNA (snRNA) level and presented as mean ±SD (*P<0.05).

FIG. 7a illustrates putative binding sites for miR-376c-3p at full-length 3′UTR (top) and truncated 3′UTR (bottom) of mouse Atrogin-1. Only regions conserved between human and mouse are contained.

FIG. 7b illustrates confirmation of effects of miR-376c-3p on wild type (WT) Atrogin-1 3′UTR or deletion-mutated (Mut) Atrogin-1 3′UTR by measuring activity of luciferase reporters with WT or mutant 3′UTR (***P<0.001).

FIG. 7c illustrates pull-down analysis results obtained by using ASO (with or without biotin) and streptavidin beads. The quantitative analysis was performed by qRT-PCR (**P<0.01).

FIG. 8 illustrates pull-down analysis results for Atrogin-1 3′UTR obtained by using streptavidin beads in C2C12 cells. Data were presented as mean ±SD of 3 independent experiments.

FIGS. 9a and 9b illustrate immunoblot assay results for Atrogin-1 in differentiated primary myoblasts (FIG. 9a) and HSMIMs (FIG. 9b) which were transfected with miR-376C-3p or inhibitor (I)-miR-376C-3p. Expression levels of Atrogin-1 were quantified using ImageJ software and normalized with ACTB.

FIG. 9c illustrates immunoblot assay results for Atrogin-1 in C2C12 cells transfected with miR-376C-3p or inhibitor (I)-miR-376C-3p. The results were normalized with ACTB expression.

FIG. 10a illustrates images of differentiated muscle cells expressing miR-376c-3p or a control (wild type, Ctrl) (green, MyHC; blue, DAPI; scale bar, 50 μm).

FIG. 10b illustrates a quantification graph for percentages of fiber diameter in differentiated muscle cells expressing miR-376c-3p or a control (wild type, Ctrl).

FIG. 10c illustrates a quantification graph for averages of fiber diameter in differentiated muscle cells expressing miR-376c-3p or a control (wild type, Ctrl) (**P<0.01).

FIG. 10d illustrates protein proportions, which are normalized with a genomic DNA content, in differentiated HSMMs transfected with miR-376c-3p or a control (**P<0.01).

FIG. 10e illustrates a plan for adenoviral injection into TA muscles (AdmiRa-376c-3p) and control TA muscles (AdmiRa-Ctrl) of 23-month-old mice.

FIG. 11a illustrates immunoblot assay results for Atrogin-1 in C2C12 myotubes infected with AdmiRa-376c-3p or a control (AdmiRa-Ctr1). On day 3 after differentiation of C2C12 cells into myotubes, muscle cells were infected with AdmiRa-376c-3p or the control (AdmiRa-Ctr1). Twenty-four hours after infection with adenovirus, expression of Atrogin-1 was measured through Western blotting and normalized with results of ACTB.

FIG. 11b illustrates fluorescent images, on day 7 after infection, of 23-month-old TA muscle tissue which was infected with GFP-labeled AdmiRa-376c-3p or a control (scale bar, 1 mm).

FIGS. 12a to 12c illustrate graphs for images of virus-infected muscle cross-section (FIG. 12a), percentages thereof (FIG. 12b), and averages thereof (FIG. 12c) (red, laminin; blue, DAPI; scale bar, 50 μm; *P<0.05; ** P<0.01).

FIG. 12d illustrates immunoblot assay results for Atrogin-1 and eIF3f in AdmiRa-Ctrl- or 376c-3p-infected TA muscle tissue of 23-month-old mice.

FIG. 12e illustrates relative expression levels of Atrogin-1 and eIF3f using ImageJ in the immunoblot assay results of FIG. 12d. The results were normalized with a mean level of ACTN1. Data were presented as mean ±SD (*P<0.05, **P<0.01.) FIG. 12f illustrates results obtained by analyzing TA muscle fatigue in young or old mice.

FIG. 12g illustrates results obtained by analyzing TA muscle fatigue in adenovirus-infected old mice.

FIG. 13a illustrates images of C2C12 myotubes transfected with miR-376c-3p or si-Atrogin-1. 24-hour treatment with 100 μM dexamethasone (dex) was performed or was not performed (green, MyHC; blue, DAPI; scale bar, 50 μm).

FIGS. 13b and 13c graphically illustrate percentages (b) and averages (c) of quantified diameters of muscle fibers of FIG. 13a. Four different images were randomly selected for myotube diameter measurement (*P<0.05, **P<0.01).

FIG. 13d illustrates immunoblot assay results for Atrogin-1.

FIG. 13e illustrates relative protein proportions, which are normalized with a genomic DNA content, in C2C12 myotubes transfected with miR-376c-3p or si-Atrogin-1 (*P<0.05, **P<0.01).

FIG. 14a illustrates images of normal C2C12 myotubes or C2C12 myotubes with inhibited Atrogin-1 expression (green, MyHC; blue, DAPI; scale bar, 50 μm).

FIGS. 14b and 14c graphically illustrate percentages (FIG. 14b) and averages (FIG. 14c) of quantified myotube diameters. Four different images were randomly selected for myotube diameter measurement (*P<0.05).

FIG. 15a illustrates images of C2C12 myotubes transfected with miR-376c-3p or a control which were cultured with or without colon-26 (C26) cultured media (CM) (green, MyHC; blue, DAPI; scale bar, 50 μm).

FIGS. 15b and 15c illustrate percentages (FIG. 15b) and averages (FIG. 15c) of quantified fiber diameters. Three images were randomly selected for myotube diameter measurement (*P<0.05).

FIG. 15d illustrates immunoblot assay results for Atrogin-1 and eIF3f in C2C12 muscle cells transfected with miR-376c-3p depending on presence or absence of CM. Relative expression levels of Atrogin-1 and eIF3f proteins were measured using ImageJ. The results were normalized with GAPDH expression.

FIG. 15e illustrates a schematic representation for a process of injecting adenovirus into TA muscle tissue of C26 tumor-bearing mice (n=6). On days 7 and 10 after inoculation of 8-week-old mice with C26 tumor cells, AdmirRa-376c-3p or AdmiRa-Control (10⁸ CFU/50 μl/injection) was respectively injected into one TA muscle and a contralateral muscle thereof.

FIG. 16a illustrates body weights measured before inoculation with C26 tumor and on day 14 after inoculation with C26 tumor.

FIG. 16b illustrates a ratio of TA muscle weight to tibia length in tumor-inoculated mice (cachexia-induced group) and non-tumor-inoculated mice (normal). Data were presented as mean ±SD (n=6; *P<0.05, **P<0.01)

FIG. 17a illustrates percentages of weight of TA muscle tissue infected with AdmiRa-376c-3p or control virus on day 14 after tumor injection. Data were normalized with tibia length (**P<0.01).

FIGS. 17b and 17c illustrate morphological analysis results for cross section area (CSA). Six images were randomly selected using ImageJ software for CSA measurement, and measurement was performed. Data were presented as mean ±SD (*P<0.05, **P<0.01).

FIG. 18 illustrates an Atrogin-1 protein expression regulation model mediated by miRNAs located in the Dlk1-Dio3 cluster with aging. Atrogin-1 protein expression levels increased due to overall decreased expression of miRNAs in a Dlk1-Dio3 genome region of old muscle tissue. Increased expression of Atrogin-1 with aging may induce degradation of target proteins such as eIF3f, and thus may lead to muscular atrophy in aged muscles. It has been elucidated that this series of events is an important underlying mechanism for development of aging-induced sarcopenia.

FIG. 19a illustrates results of correlation analysis between a human age and expression of miR-23a-3p. The results were quantified by qRT-PCR. In addition, the results were evaluated using Spearman's correlation test (p; 95% CI). FIG. 19b illustrates relative expression of miR-23a-5p, miR-23a-3p, miR-19a-3p, and miR-19b-3p in TA muscles with aging.

### BEST MODE

In an aspect, the present invention provides a pharmaceutical composition for preventing or treating a muscular disease, comprising, as an active ingredient, a miRNA located in Dlk1-Dio3 cluster or a variant thereof.

In the present invention, the miRNA located in the Dlk1-Dio3 cluster may be any one selected from the group consisting of miRNA-668 (SEQ ID NO: 1), miRNA-376c (SEQ ID NO: 2), miRNA-494 (SEQ ID NO: 4), miRNA-541 (SEQ ID NO: 5), miRNA-377 (SEQ ID NO: 10), miRNA-1197 (SEQ ID NO: 6), miRNA-495 (SEQ ID NO: 7), miRNA-300 (SEQ ID NO: 14), miRNA-409 (SEQ ID NO: 16), miRNA-544a (SEQ ID NO: 18), miRNA-379 (SEQ ID NO: 19), miRNA-431 (SEQ ID NO: 23), miRNA-543 (SEQ ID NO: 30), and miRNA-337 (SEQ ID NO: 36).

As used herein, the term “Dlk1-Dio3 cluster” is an abbreviation of “delta-like 1 homolog-type 3 iodothyronine deiodinase” and is known as the largest miRNA cluster.

As used herein, the term “miRNA” refers to a non-coding RNA of about 21 to 24 nucleotides, which is transcribed from DNA but not translated into a protein. The miRNA is processed from a primary transcript known as pri-miRNA to a short stem-loop structure termed pre-miRNA and finally to a functional miRNA. During maturation, each pre-miRNA provides two unique fragments with high complementarity, one of the fragments originating from a 5 ‘arm of a gene encoding the pri-miRNA, and the other originating from a 3’ arm of a gene encoding the pri-miRNA. A mature miRNA molecule is partially complementary to one or more messenger RNAs (mRNAs), and a main function thereof is to down-regulate gene expression.

According to the international nomenclature for miRNAs, unique names having a predetermined format are assigned as follows.

A mature miRNA is named in a format of “sss-miR-X-Y”, where “sss” is a three-letter code representing a species of the miRNA and, for example, may be “hsa” which symbolizes a human. In miR, the upper case “R” indicates that the miRNA refers to a mature miRNA. Xis any unique number assigned to sequences of miRNAs in a particular species. In a case where several highly-homologous miRNAs are known, a letter may follow the number. For example, “376a” and “376b” refer to highly-homologous miRNAs. Y indicates whether a mature miRNA obtained by cleavage of a pre-miRNA corresponds to a 5′ arm of a gene encoding the pri-miRNA (in this case, Y is “-5p”) or a 3′ arm thereof (in this case, Y is “-3p”).

Among the miRNAs located in the Dlk1-Dio3 region which are mentioned in the present invention, referring to “hsa-miR-376c-3p” as an example, “hsa” means that the miRNA pertains to a human miRNA, “miR” refers to a mature miRNA, “376” refers to any number assigned to this particular miRNA, and “3p” means that the mature miRNA has been obtained from a 3′ arm of a gene encoding a pri-miRNA.

Meanwhile, in the present invention, the miRNAs located in the Dlk1-Dio3 region may be miRNAs corresponding to -3p and/or -5p. In an embodiment, miRNA-376c in the present invention may be miR-376c-3p or miRNA376c-5p.

As used herein, the term “miRNA variant” may be a miRNA having a base sequence that maintains a homology of 90% or more, more particularly 95% or more, and even more particularly 98%, to a miRNA (SEQ ID NO: 1, 2, 4, 5, 6, 7, 10, 14, 16, 18, 19, 23, 30, or 36) located in the Dlk1-Dio3 cluster according to the present invention. In the present invention, the miRNA variant may be a miRNA fragment.

As used herein, the term “miRNA fragment” may include, in a case of being compared with a miRNA reference sequence, a sequence with deletion or a segment of the same sequence segment as the reference sequence at a corresponding position. The “reference sequence” means a sequence designated to be used as a basis for sequence comparison. In the present invention, the miRNA reference sequence may be a polynucleotide having a sequence of SEQ ID NO: 1, 2, 4, 5, 6, 7, 10, 14, 16, 18, 19, 23, 30, or 36.

As used herein, the term “miRNA mimic” refers to a polynucleotide that mimics miRNA action, and the mimic can be therapeutically targeted.

A miRNA located in the Dlk1-Dio3 cluster can decrease an expression level of Atrogin-1/MAFbx protein. In addition, the miRNA located in the Dlk1-Dio3 cluster is capable of directly interacting with 3′-untranslated region (3′-UTR) of a polynucleotide encoding the Atrogin-1/MAFbx protein, and suppressing expression of Atrogin-1/MAFbx which is a muscle-degrading enzyme.

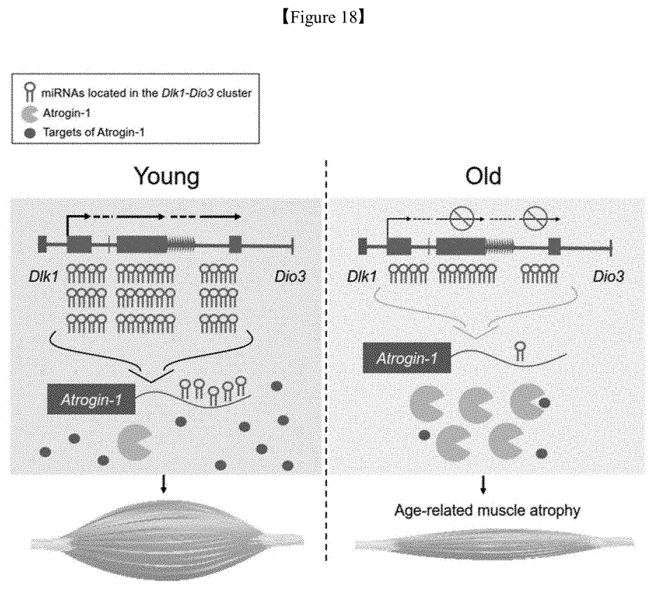

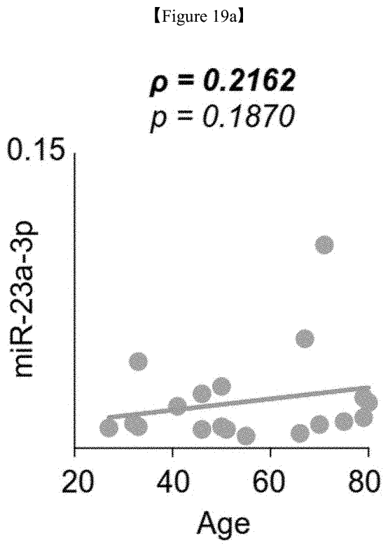

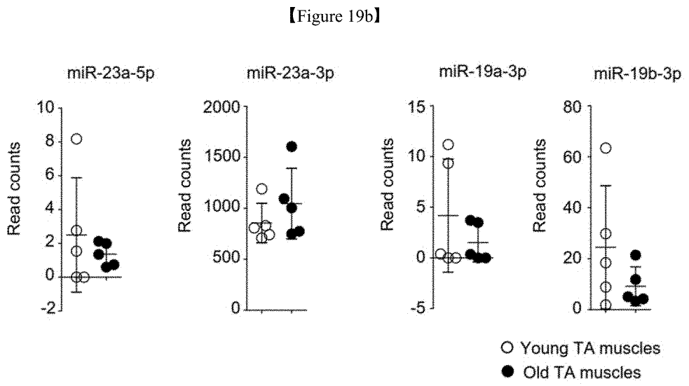

As used herein, the term “Atrogin-1” refers to one of the representative muscle-pecific E3 ligases such as MuRF1. Unlike other muscular disease, a role of Atrogin-1 in muscle aging is relatively less known. In an embodiment of the present invention, in muscle tissue of old mice, a protein level of Atrogin-1 significantly increases, whereas there was no change in a gene expression level thereof. This means that the miRNA regulates expression of Atrogin-1 in a post-transcriptional manner (see FIG. 18). According to studies to date, it is known that miR-19a, miR-19b, and miR-23a target Atrogin-1 to lead to muscle hypertrophy. However, in the present invention, these miRNAs were not differentially expressed in young muscle tissue and old muscle tissue (see FIGS. 19a and 19b). In view of this, it can be seen that the miRNA in the Dlk-1-Dio3 cluster, of which expression decreases with aging, may be an important internal factor in Atrogin-1-mediated aging-induced sarcopenia.

As used herein, the term “muscular disease” refers to all diseases that may develop due to weakened muscle strength, and examples thereof include, but not limited to, sarcopenia, muscular atrophy, muscle dystrophy, or acardiotrophia. Specifically, the “sarcopenia” may be aging-induced sarcopenia.

The “weakened muscle strength” means a state in which strength of one or more muscles is decreased. The weakened muscle strength may be limited to any one muscle, a portion of a body, upper limb, lower limb, or the like, or may appear throughout the body. In addition, subjective symptoms of weakened muscle strength, including muscle fatigue and myalgia, can be quantified in an objective way through medical examinations. Causes for weakened muscle strength include, but not limited to, muscle damage, decreased muscle mass due to decreased differentiation of muscle cells, and muscle aging.

Aging-induced sarcopenia is a muscular disease in which skeletal muscles that make up arms, legs, and the like are greatly decreased, and which is caused by decrease in muscle cells due to aging and lack of activity. Sarcopenia is a compound word of “sarco”, which means muscle, and penia, which means lack or decrease. In early 2017, the World Health Organization (WHO) recognized, as an official disease, a state with less muscle mass than normal, and assigned a disease classification code to aging-induced sarcopenia.

Muscular atrophy is a disease in which muscles shrink and muscles of arms and legs gradually shrink in an almost symmetrical manner. There are various forms of muscular atrophy. Amyotrophic lateral sclerosis and progressive spinal amyotrophy are most common. Both diseases are due to progressive denaturation of motor nerve fibers and cells in spinal cord, but causes thereof are unclear. Specifically, amyotrophic lateral sclerosis is also referred to as “Lou Gehrig's disease” and is a disease in which motor cells in spinal cord or diencephalon are gradually destroyed and muscles under control of these cells shrink, thereby making it impossible to exert strength. In progressive spinal amyotrophy, degeneration of pyramidal tract is not exhibited, and degeneration of spinal cord anterior horn cells progresses in a chronic manner.

Muscle regressive atrophy is a disease in which gradual muscular atrophy and muscle weakness are manifested, and means, in a pathological sense, a degenerative myopathy characterized by necrosis of muscle fibers. In muscle regressive atrophy, muscle cell membrane damage causes muscle fibers to go through necrosis and degeneration processes, so that weakened muscle strength and atrophy occur. Muscle regressive atrophy can be divided into sub-diseases depending on extent and distribution of weakened muscle, age of onset, rate of progression, severity of symptoms, and family history. Non-limiting examples of such muscle regressive atrophy include Duchenne muscular dystrophy, Becker muscular dystrophy, limb-girdle muscular dystrophy, Emery-Dreifuss muscular dystrophy, facioscapulohumeral muscular dystrophy, myotonic muscular dystrophy, oculopharyngeal muscular dystrophy, distal muscular dystrophy, and congenital muscular dystrophy.

Acardiotrophia is a condition in which a heart gets to shrink due to external or internal factors. In a case of starvation, wasting disease, or senility, acardiotrophia may lead to brown atrophy symptoms of heart which cause myocardial fibers to become lean and thin, and thus result in decreased adipose tissue.

In another aspect, the present invention provides a pharmaceutical composition for preventing or treating a muscular disease, comprising, as an active ingredient, a vector loaded with a nucleotide encoding a miRNA located in the Dlk1-Dio3 cluster or a variant thereof. Here, the miRNA located in Dlk1-Dio3 cluster is as described above.

The vector may include, but not limited to, a plasmid vector, a cosmid vector, a virus, and analogs thereof. Specifically, the virus may be any one or more selected from the group consisting of adenovirus, adeno-associated virus, herpes simplex virus, lentivirus, retrovirus, and poxvirus. More specifically, the virus may be, but not limited to, adenovirus or adeno-associated virus. The vector loaded with a nucleotide encoding the miRNA located in the Dlk1-Dio3 cluster or a variant thereof can be produced by a cloning method known in the art, and production thereof is not particularly limited to such a method.

Meanwhile, a preferred dose of the pharmaceutical composition according to the present invention for preventing or treating a muscular disease, comprising, as an active ingredient, the vector loaded with the nucleotide encoding the miRNA located in the Dlk1-Dio3 cluster or a variant thereof varies depending on condition and body weight of an individual, severity of disease, form of drug, route of administration, and duration, and may be appropriately selected by those skilled in the art. Specifically, a patient may be administered with virus particles, infectious virus units (TCID₅₀), or plaque forming units (pfu) of 1×10⁵ to lx10¹⁸. Preferably, the patient may be administered with virus particles, infectious virus units, or plaque forming units of 1×10⁵, 2×10⁵, 5×10⁵, 1×10⁶, 2×10⁶, 5×10⁶, 1×10⁷, 2×10⁷, 5×10⁷, 1×10⁸, 2×10⁸, 5×10⁸, 1×10⁹, 2×10⁹, 5×10⁹, 1×10¹⁰, 5×10¹⁰, 1×10¹¹, 5×10¹¹, 1×10¹², 1×10¹³, 1×10¹⁴, 1×10¹⁵, 1×10¹⁶, 1×10¹⁷, or more, and various values and ranges can be included therebetween. In addition, a dose of virus may 0.1 ml, 1 ml, 2 ml, 3 ml, 4 ml, 5 ml, 6 ml, 7 ml, 8 ml, 9 ml, 10 ml, or more, and can include all values and ranges therebetween.

In yet another aspect, the present invention provides a pharmaceutical composition for preventing or treating cachexia, comprising, as an active ingredient, a miRNA located in Dlk1-Dio3 cluster or a variant thereof In the present invention, the miRNA located in the Dlk1-Dio3 cluster and the variant thereof are as described above.

As used herein, the term “cachexia” refers to a high degree of symptoms of systemic weakness which can be seen at a terminal stage of cancer, tuberculosis, hemophilia, or the like. Cachexia is considered to be a kind of poisoning state caused by various organ disorders throughout a body. Symptoms including muscle weakness, rapid emaciation, anemia, lethargy, and skin yellowing occur. Underlying diseases for cachexia include malignant tumor, Basedow's goiter, hypopituitarism, and the like. It has been found that biologically active substances such as tumor necrosis factor (TNF) produced by macrophages are also factors which exacerbate cachexia.

In the pharmaceutical composition according to the present invention for preventing or treating a muscular disease or cachexia, comprising, as an active ingredient, the miRNA located in the Dlk1-Dio3 cluster or a variant thereof, the active ingredient can be contained in any amount (effective amount) depending on uses, formulations, purposes of blending, and the like as long as the active ingredient can exhibit activity. A typical effective amount can be determined within a range of 0.001% by weight to 20.0% by weight based on a total weight of the composition. Here, the term “effective amount” refers to an amount of the active ingredient which is capable of inducing therapeutic effects on the muscular disease or cachexia. Such an effective amount can be determined experimentally within the scope of ordinary skill of those skilled in the art.

In addition, the pharmaceutical composition according to the present invention for preventing or treating a muscular disease or cachexia may further comprise a pharmaceutically acceptable carrier. As the pharmaceutically acceptable carrier, any carrier can be used as long as the carrier is a non-toxic substance suitable for delivery to a patient. Distilled water, alcohol, fat, wax, and an inactive solid may be contained as the carrier. A pharmacologically acceptable adjuvant (buffer or dispersant) may also be contained in the pharmaceutical composition.

Specifically, the pharmaceutical composition of the present invention comprises a pharmaceutically acceptable carrier in addition to an active ingredient, so that the pharmaceutical composition can be prepared into a parenteral formulation depending on route of administration by a conventional method known in the art. Here, the term “pharmaceutically acceptable” means that the carrier does not have more toxicity than a subject to be applied (prescribed) can adapt while not suppressing activity of the active ingredient.

In a case where the pharmaceutical composition according to the present invention for preventing or treating a muscular disease or cachexia is prepared into a parenteral formulation, the pharmaceutical composition can be formulated in the form of an injection, an agent for transdermal administration, a nasal inhalant, and a suppository, together with a suitable carrier, according to methods known in the art. In a case of being formulated into an injection, as a suitable carrier, sterilized water, ethanol, polyol such as glycerol and propylene glycol, or a mixture thereof may be used. Specifically, Ringer's solution, phosphate buffered saline (PBS) containing triethanolamine or sterilized water for injection, an isotonic solution such as 5% dextrose, or the like may be used.

A dose of the pharmaceutical composition according to the present invention for preventing or treating a muscular disease or cachexia may be in a range of 0.01 ug/kg to 10 g/kg per day, and, particularly, in a range of 0.01 mg/kg to 1 g/kg per day, depending on condition, body weight, gender, or age of a patient, severity of the patient, or route of administration. Administration can be performed once or several times a day. Such a dose should not be construed in any way as limiting the scope of the present invention.

In addition, the present invention provides a pharmaceutical composition for preventing or treating cachexia, comprising, as an active ingredient, a vector loaded with a nucleotide encoding a miRNA located in Dlk1-Dio3 cluster or a variant thereof. Here, the miRNA located in the Dlk1-Dio3 cluster is as described above.

As described above, the vector may include, but not limited to, a plasmid vector, a cosmid vector, a virus, and analogs thereof. Specifically, the virus may be any one or more selected from the group consisting of adenovirus, adeno-associated virus, herpes simplex virus, lentivirus, retrovirus, and poxvirus. More specifically, the virus may be, but not limited to, an adenovirus. The vector loaded with a nucleotide encoding the miRNA located in the Dlk1-Dio3 cluster or a variant thereof can be produced by a cloning method known in the art, and production thereof is not particularly limited to such a method.

Meanwhile, a preferred dose of the pharmaceutical composition according to the present invention for preventing or treating cachexia, comprising, as an active ingredient, the vector loaded with the nucleotide encoding the miRNA located in the Dlk1-Dio3 cluster or a variant thereof varies depending on condition and body weight of an individual, severity of disease, form of drug, route of administration, and duration, and may be appropriately selected by those skilled in the art. Specifically, a patient may be administered with virus particles, infectious virus units (TCID₅₀), or plaque forming units (pfu) of 1×10⁵ to lx10¹⁸. Preferably, the patient may be administered with virus particles, infectious virus units, or plaque forming units of 1×10⁵, 2×10⁵, 5×10⁵, 1×10⁶, 2×10⁶, 5×10⁶, 1×10⁷, 2×10⁷, 5×10⁷, 1×10⁸, 2×10⁸, 5×10⁸, 1×10⁹, 2×10⁹, 5×10⁹, 1×10¹⁰, 5×10¹⁰, 1×10¹¹, 5×10¹¹, 1×10¹², 1×10¹³, 1×10¹⁴, 1×10¹⁵, 1×10¹⁶, 1×10¹⁷, or more, and various values and ranges can be included therebetween. In addition, a dose of virus may 0.1 ml, 1 ml, 2 ml, 3 ml, 4 ml, 5 ml, 6 ml, 7 ml, 8 ml, 9 ml, 10 ml, or more, and can include all values and ranges therebetween.

In addition, the present invention provides a method for preventing or treating a muscular disease, comprising a step of administering to a subject a pharmaceutical composition according to the present invention for preventing or treating the muscular disease.

In addition, the present invention provides a method for preventing or treating cachexia, comprising a step of administering to a subject a pharmaceutical composition according to the present invention for preventing or treating cachexia.

The subject may be a mammal, in particular, a human, but is not limited thereto. In addition, the administration may be carried out through any one route selected from the group consisting of intravenous, intramuscular, intradermal, subcutaneous, intraperitoneal, intranasal, intrapulmonary, rectal, intraarteriolar, intraventricular, intralesional, intrathecal, local, and combinations thereof. A mode of administration may vary depending on a type of a drug to be administered.

In addition, the present invention provides a use of a miRNA located in Dlk1-Dio3 cluster or a variant thereof for the manufacture of a medicament for preventing or treating a muscular disease, and provides a use of a vector loaded with a nucleotide encoding a miRNA located in Dlk1-Dio3 cluster or a variant thereof for the manufacture of a medicament for preventing or treating a muscular disease.

In addition, the present invention provides a use of a miRNA located in Dlk1-Dio3 cluster or a variant thereof for the manufacture of a medicament for preventing or treating cachexia, and provides a use of a vector loaded with a nucleotide encoding a miRNA located in Dlk1-Dio3 cluster or a variant thereof for the manufacture of a medicament for preventing or treating cachexia.

### Mode for Invention

Hereinafter, the present invention will be described in more detail with reference to the following examples. However, the following examples are provided to merely illustrate the present invention, and the scope of the present invention is not limited thereto.

### EXAMPLE 1

### Sample Preparation

Human skeletal muscles (gluteus maximus muscles) obtained from patients who underwent total hip replacement arthroplasty (THRA) at the Seoul National University Bundang Hospital (SNUBH) were immediately transferred to liquid nitrogen and stored at −70° C. The SNUBH's Institutional Review Board (B-1710-050-009) approved the present experiment. Written consents were obtained from participants or legal guardians, and a total of 20 patient samples (at 25, 27, 32, 33 (2 patient samples), 41, 46 (2 patient samples), 50 (2 patient samples), 51, 55, 66, 67, 70, 71, 75, 79 (2 patient samples), and 80 years old) were used to evaluate expression of miRNA or Atrogin-1 protein.

All 20 samples were used for miRNA expression assay. However, only 8 samples were available for immunoblotting due to a limited amount of solubility. RNAs and proteins were isolated and purified from human samples of 30 μg or less. In order to analyze miRNA expression, the RNAs were further purified using TRIzol (Invitrogen). For immunoblot assay, muscle tissue was homogenized using T 10 Basic Ultra-Turrax Disperser (IKA, China) and then lysed using PRO-PREP (iNtRON Biotech).

### EXAMPLE 2

### Animal Model

Young C57BL/6 mice (3 months old) and old C57BL/6 mice (24 months old) were purchased from the Laboratory Animal Resource Center (at the Korea Research Institute of Bioscience and Biotechnology (KRIBB)). BALB/c (6-week-old) mice were purchased from Damul Science (Daej eon, Korea). All mice in the present study were kept on a standard experimental diet (3.1 kcal/g) using feeds purchased from Damul Science (Daej eon, Korea). In order to over-express miRNA mimics in muscle tissue, 50 μl (10⁸ CFU) of adenovirus, AdmiRa-376c-3p, or a control (Applied Biological Materials Inc, Canada) were respectively injected into TA muscle or a contralateral muscle thereof in the young mice and the old mice.

The injection was performed once a week using a 29G (0.33 mm) insulin syringe. Four weeks after injection, muscle tissue was isolated from the adenovirus-injected mice and used for analysis. In order to create a cachexia mouse model, BABL/c mice were subcutaneously injected with C26 cells (5×10⁵ cells in 50 μl of PBS) using an insulin syringe. On days 7 and 10 after tumor inoculation, 50 μl (10⁸ CFU) was injected intramuscularly into TA muscle or a contralateral muscle thereof in tumor-bearing mice.

Colon 26 cells (CLS Cell Lines Service) were cultured in RPMI1640 (Gibco) containing amphotericin B-penicillin-streptomycin and 10% FBS. Mouse and virus experiments were performed according to protocols approved by the KRIBB's Animal Care and Use Committee.

### EXAMPLE 3

### Cell Culture

Primary myoblasts were isolated from hind leg muscles of the mice in Example 2. Muscle tissue was finely sliced with scissors, and then placed in a dissociation buffer containing dispase II (2.4 U/mL, Roche), collagenase D (1%, Roche), and 2.5 μM CaCl₂ followed by incubation at 37° C. for 20 minutes. The slurry was ground using a serologic pipette and passed through a 70 μm nylon mesh (BD Biosciences) to remove debris.

The cells were collected and cultured in Ham's F-10 (Gibco) with 20% FBS containing amphotericin B-penicillin-streptomycin and 5 ng/mL of bFGF. In order to remove fibroblasts, the cells were smeared on an uncoated plate for 1 hour, and the immobilized cells were transferred to a collagen-coated culture dish. Differentiation of primary muscle fibers was induced by culturing the cells in DMEM (Gibco) differentiation medium containing antibiotics and 5% horse serum. C2C12 cells (ATCC) were cultured in DMEM (Gibco) containing amphotericin B-penicillin-streptomycin and 10% FBS. The medium was replaced with a differentiation medium, so that differentiation was initiated 24 hours or 48 hours after smearing of the cells.

For dexamethasone-caused atrophy, C2C12 cells were initially differentiated for 4 days and then 100 μM dexamethasone (Sigma-Aldrich) was added to the medium. Human skeletal muscle (Lonza) was obtained from a 17-year-old donor and cultured in skeletal muscle basal medium 2 (Lonza) containing gentamicin-amphotericin B, human epidermal growth factor (hEGF), dexamethasone, L-glutamine, and 10% FBS. After 24 to 48 hours, differentiation was initiated and cultured in DMEM/F12 (Gibco) containing gentamicin-amphotericin B and 2% horse serum.

For colon 26 (C26) conditioned media, colon 26 cultured in media consisting of DMEM (Gibco) with 10% fetal bovine serum. After 72 h, the supernatant was collected and filtered through a 0.22 micron filter. C26 culture medium treatment was 50% in differentiation medium (DMEM with 2% horse serum).

### EXAMPLE 4

### Transfection and Luciferase Assay

Mimics and inhibitors for miRNAs were purchased from mirVana (Invitrogen) or AccuTarget™ (Bioneer) (see Tables 1 and 2 below). Information on siRNAs was additionally added to Table 3 below. Primary myoblasts, C2C12, or human skeletal muscle myoblasts were transfected with mimics and inhibitors for miRNAs and siRNAs (50 nM to 100 nM for each) using RNAiMAX (Invitrogen).

TABLE 1

| miRNA | Assay ID | Cat. NO |
|---|---|---|
| Negative control | — | AM17121 |
| 376c-3p | 002450 | MC12548 |

TABLE 2

| | | SEQ ID | Accession |
|---|---|---|---|
| miRNA | Sequence | NO. | NO. |
| 493 | ctggcctccagggattgtacatggtaggattcattcattcgtttgcacattcggtg | 21 | MI0003132 |
| | aaggtctactgtgtgccaggccctgtgccag | | |
| 337 | gtagtcagtagttggggggtgggaacggcttcatacaggagttgatgcacagtt | 36 | MI0000806 |
| | atccagctcctatatgatgcctttcttcatccccttcaa | | |
| 665 | tctcctcgaggggtctctgcctctacccaggactattcatgaccaggaggctga | 26 | MI0005563 |
| | ggcccctcacaggcggc | | |
| 431 | tcctgcttgtcctgcgaggtgtcttgcaggccgtcatgcaggccacactgacggt | 23 | MI0001721 |
| | aacgttgcaggtcgtcttgcagggcttctcgcaag | | |
| | acgacatcctcatcaccaacgacg | | |
| 433 | ccggggagaagtacggtgagcctgtcattattcagagaggctagatcctctgtgt | 38 | MI0001723 |
| | tgagaaggatcatgatgggctcctcggtgttctccagg | | |
| 432 | tgactcctccaggtcttggagtaggtcattgggtggatcctctatttccttacgtgg | 20 | MI0003133 |
| | gccactggatggctcctccatgtcttggagtagatca | | |
| 370 | agacagagaagccaggtcacgtctctgcagttacacagctcacgagtgcctgct | 28 | MI0000778 |
| | ggggtggaacctggtctgtct | | |
| 379 | agagatggtagactatggaacgtaggcgttatgatttctgacctatgtaacatggt | 19 | MI0000787 |
| | ccactaactct | | |
| 299 | aagaaatggtttaccgtcccacatacattttgaatatgtatgtgggatggtaaacc | 15 | MI0000744 |
| | gcttctt | | |
| 380 | aagatggttgaccatagaacatgcgctatctctgtgtcgtatgtaatatggtccac | 13 | MI0000788 |
| | atctt | | |
| 1197 | acttcctggtatttgaagatgcggttgaccatggtgtgtacgctttatttgtgacgta | 6 | MI0006656 |
| | ggacacatggtctacttcttctcaatatca | | |
| 323a | ttggtacttggagagaggtggtccgtggcgcgttcgctttatttatggcgcacatt | 29 | MI0000807 |
| | acacggtcgacctctttgcagtatctaatc | | |
| 758 | gcctggatacatgagatggttgaccagagagcacacgctttatttgtgccgtttgt | 12 | M10003757 |
| | gacctggtccactaaccctcagtatctaatgc | | |
| 329 | ggtacctgaagagaggttttctgggtttctgtttctttaatgaggacgaaacacac | 11 | MI0001725 |
| | ctggttaacctcttttccagtatc | | |
| 494 | gatactcgaaggagaggttgtccgtgttgtcttctctttatttatgatgaaacataca | 4 | MI0003134 |
| | cgggaaacctcttttttagtatc | | |
| 1193 | gtagctgaggggatggtagaccggtgacgtgcacttcatttacgatgtaggtca | 35 | MI0014205 |
| | cccgtttgactatccaccagcgcc | | |
| 543 | tacttaatgagaagttgcccgtgtttttttcgctttatttgtgacgaaacattcgcggt | 30 | MI0005565 |
| | gcacttctttttcagtatc | | |
| 495 | tggtacctgaaaagaagttgcccatgttattttcgctttatatgtgacgaaacaaac | 7 | MI0003135 |
| | atggtgcacttctttttcggtatca | | |
| 376c | aaaaggtggatattccttctatgtttatgttatttatggttaaacatagaggaaattcc | 2 | MI0000776 |
| | acgtttt | | |
| 654 | gggtaagtggaaagatggtgggccgcagaacatgtgctgagttcgtgccatat | 27 | MI0003676 |
| | gtctgctgaccatcacctttagaagccc | | |
| 376b | cagtccttctttggtatttaaaacgtggatattccttctatgtttacgtgattcctgg | 25 | MI0002466 |
| | ttaatcatagaggaaaatccatgttttcagtatcaaatgctg | | |
| 376a | taaaaggtagattctccttctatgagtacattatttatgattaatcatagaggaaaat | 3 | MI0000784 |
| | ccacgttttc | | |
| 300 | tgctacttgaagagaggtaatccttcacgcatttgctttacttgcaatgattatacaa | 14 | MI0005525 |
| | gggcagactctctctggggagcaaa | | |
| 381 | tacttaaagcgaggttgccctttgtatattcggtttattgacatggaatatacaagg | 33 | MI0000789 |
| | gcaagctctctgtgagta | | |
| 487b | ttggtacttggagagtggttatccctgtcctgttcgttttgctcatgtcgaatcgtac | 24 | MI0003530 |
| | agggtcatccactttttcagtatcaa | | |
| 539 | atacttgaggagaaattatccttggtgtgttcgctttatttatgatgaatcatacaag | 32 | MI0003514 |
| | gacaatttctttttgagtat | | |
| 544a | attttcatcacctagggatcttgttaaaaagcagattctgattcagggaccaagatt | 18 | MI0003515 |
| | ctgcatttttagcaagttctcaagtgatgctaat | | |
| 382 | tacttgaagagaagttgttcgtggtggattcgctttacttatgacgaatcattcacg | 8 | MI0000790 |
| | gacaacacttttttcagta | | |
| 134 | cagggtgtgtgactggttgaccagaggggcatgcactgtgttcaccctgtgggc | 17 | MI0000474 |
| | cacctagtcaccaaccctc | | |
| 668 | ggtaagtgcgcctcgggtgagcatgcacttaatgtgggtgtatgtcactcggctc | 1 | MI0003761 |
| | ggcccactacc | | |
| 485 | acttggagagaggctggccgtgatgaattcgattcatcaaagcgagtcatacac | 40 | MI0002469 |
| | ggctctcctctcttttagt | | |
| 154 | gtggtacttgaagataggttatccgtgttgccttcgctttatttgtgacgaatcatac | 34 | MI0000480 |
| | acggttgacctatttttcagtaccaa | | |
| 496 | cccaagtcaggtactcgaatggaggttgtccatggtgtgttcattttatttatgatga | 31 | MI0003136 |
| | gtattacatggccaatctcctttcggtactcaattcttcttggg | | |
| 377 | ttgagcagaggttgcccttggtgaattcgctttatttatgttgaatcacacaaaggc | 10 | MI0000785 |
| | aacttttgtttg | | |
| 541 | acgtcagggaaaggattctgctgtcggtcccactccaaagttcacagaatgggt | 5 | M10005539 |
| | ggtgggcacagaatctggactctgcttgtg | | |
| 409 | tggtactcggggagaggttacccgagcaactttgcatctggacgacgaatgttg | 16 | MI0001735 |
| | ctcggtgaaccccttttcggtatca | | |
| 412 | ctggggtacggggatggatggtcgaccagttggaaagtaattgtttctaatgtac | 9 | MI0002464 |
| | ttcacctggtccactagccgtccgtatccgctgcag | | |
| 369 | ttgaagggagatcgaccgtgttatattcgctttattgacttcgaataata | 37 | MI0000777 |
| | catggttgatcttttctcag | | |
| 410 | ggtacctgagaagaggttgtctgtgatgagttcgcttttattaatgacgaatataac | 41 | MI0002465 |
| | acagatggcctgttttcagtacc | | |
| 127 | tgtgatcactgtctccagcctgctgaagctcagagggctctgattcagaa | 43 | MI0000472 |
| | agatcatcggatccgtctgagcttggctggtcggaagtctcatcatc | | |
| 136 | tgagccctcggaggactccatttgttttgatgatggattcttatgctccatcatcgtc | 22 | MI0000475 |
| | tcaaatgagtcttcagagggttct | | |
| 411 | tggtacttggagagatagtagaccgtatagcgtacgctttatctgtgacgtatgta | 42 | MI0003675 |
| | acacggtccactaaccctcagtatcaaatccatccccgag | | |

TABLE 3

| Target gene | Cat. NO | Sequence | SEQ ID NO |
|---|---|---|---|
| Negative control | SN-1002 | — | — |
| Atrogin-1 | 1357210 | agagagucgg | 66 |
| | | caagucugu | |
| | | (sense) | |
| | | acagacuugc | 67 |
| | | cgacucucu | |
| | | (antisense) | |
| | 1357211 | gauagaugug | 62 |
| | | uucgucuua | |
| | | (sense) | |
| | | uaagacgaac | 63 |
| | | acaucuauc | |
| | | (antisense) | |
| | 1357212 | gugaucuaag | 64 |
| | | augggaagg | |
| | | (sense) | |
| | | ccuucccauc | 65 |
| | | uuagaucac | |
| | | (antisense) | |

For luciferase assay, full-length 5598 nt 3′UTR of Atrogin-1 mRNA or a 2840 nt 3′UTR fragment thereof which contains only binding sites for miR-376c-3p conserved between human and mouse were cloned into pmirGLO (Promega). A coding sequence of vector 1uc2 (luciferase gene) was present at a multiple cloning site, and a coding sequence of vector hRluc-neo coding sequence was present as an internal control. An Atrogin-1 3′UTR mutant with deletion of a miR-376c-3p binding portion (positioned at 3781 to 3787) was also cloned into the pmirGLO vector for luciferase assay.

293T cells were transfected with 50 nM of miRNA mimics and luciferase plasmids (200 ng) using Lipofectamine 2000 (Invitrogen). 48 hours after transduction, cell lysate was subjected to assay using Dual-Luciferase Reporter Assay System (Promega) and Victor X3 (Perkin Elmer).

### EXAMPLE 5

### Quantitative RT-PCR and miRNA Expression Assay

RNA isolation and cDNA synthesis were performed according to standard protocols. Quantitative RT-PCR analysis was performed using StepOnePlus™ (Applied Biosystems) at a total reaction volume of 20 ul containing cDNA, primers, and SYBR Master Mix (Applied Biosystems). Primer sequences are shown in Table 4 below.

TABLE 4

| | | | SEQ |
|---|---|---|---|
| | | | ID |
| Species | Primer | Sequence | NO |
| Mouse | Atrogin-1 forward | ACAAGGGAAGTACGAAGGAG | 44 |
| | | CG | |
| | Atrogin-1 reverse | GGCAGTCGAGAAGTCCAGTC | 45 |
| | β-actin forward | GGCTGTATTCCCCTCCAT | 46 |
| | β-actin reverse | CCAGTTGGTAACAATGCCAT | 47 |
| | | G | |
| Human | Atrogin-1 forward | CCATCCGTCTAGTCCGCTC | 48 |
| | Atrogin-1 reverse | TGAGGTCGCTCACGAAACT | 49 |
| | | G | |
| | GAPDH forward | TGTTGCCATCAATGACCC | 50 |
| | GAPDH reverse | CCCACGACGTACTCAGCG | 51 |

Data were normalized using an mRNA expression level of ACTB or GAPDH in each reaction. For expression assay on mature microRNAs, TaqMan MicroRNA assay was performed according to the manufacturer's protocol (Applied Biosystems). An RT-qPCR reaction was carried out in a 96-well plate containing TaqMan Universal PCR Master Mix II (without Uracil-N glycosylase) and TaqMan Small RNA Assay mix. Sequences for RNA-specific primers and small RNA-specific TaqMan MGB probes used are shown in Table 5. U6 snRNA was used for normalization.

TABLE 5

| miRNA | Assay ID | Cat. NO |
|---|---|---|
| U6 snRNA | 001973 | 4427975 |
| 127-5p | 002229 | |
| 127-3p | 000452 | |
| 134-5p | 001186 | |
| 337-3p | 002157 | |
| 369-3p | 000557 | |
| 376c-3p | 002122 | |
| | 002450 | |
| 377-3p | 000566 | |
| 379-5p | 001138 | |
| 381-3p | 000571 | |
| 409-5p | 002331 | |
| 411-3p | 002238 | |
| 431-3p | 002312 | |
| 431-5p | 001979 | |
| 485-5p | 001036 | |
| 494-3p | 002365 | |
| 495-3p | 001663 | |
| 541-5p | 002200 | |
| 668-3p | 001992 | |
| | 002562 | |
| 1193-5p | 242169_mat | |
| 1197-3p | 002810 | |

### EXAMPLE 6

### Antisense Oligonucleotide (ASO) Pull-Down Analysis

For miRNA-mRNA interaction analysis, target mRNAs associated with microRNAs were purified using a hybridization-based strategy. C2C12 cells were transfected with a luciferase reporter having Atrogin-1 3′UTR that contains a wild-type or deletion-mutated miR-376c-3p binding site. Cell lysate (1 mg) was incubated at 4° C. for 3 hours and then incubated with 2μg of biotin-added ASO (see Table 6) which had been designed to specifically hybridize to Atrogin-1-3′UTR or Luciferase 2 mRNA.

TABLE 6

| Primer | Sequence | SEQ ID NO |
|---|---|---|
| mmu-miR-376c | AACATAGAGGAAATTTCAC | 52 |
| | GT | |
| U6 | TGGCCCCTGCGCAAGGATG | 53 |
| Atrogin-1 forward | CAGCTTCGTGAGCGACCTC | 54 |
| Atrogin-1 reverse | GGCAGTCGAGAAGTCCAG | 55 |
| | TC | |
| GAPDH forward | GGGAAATTCAACGGCACA | 56 |
| | GT | |
| GAPDH reverse | AGATGGTGATGGGCTTCCC | 57 |
| Luciferase 2 forward | CACCTTCGTGACTTCCCAT | 58 |
| | T | |
| Luciferase 2 reverse | TGACTGAATCGGACACAA | 59 |
| | GC | |
| 5′biotinylated ASO | ATGTGGCACTCACAGCAG | 60 |
| for endogenous | AG | |
| Atrogin-1 | | |
| 5′biotinylated ASO | AGACGGGCAAGAAAGAGG | 61 |
| for reporter mRNA | AT | |

Streptavidin-agarose beads (Novagene) were added to the combined mixture, and then further incubated at 4° C. for 2 hours. The beads were washed three times with 1 ml of NT2 buffer (50 mM Tris-HCl, pH 7.5, 150 mM NaCl, 1 mM MgCl₂, and 0.05% NP-40), and then complexes were treated with 20 units of RNase-free DNase (15 minutes, 37° C.) and 0.1% SDS/0.5 mg/ml Proteinase K (15 minutes at 55° C.) to remove DNA and protein, respectively. cDNA was synthesized from the miRNA using acid phenol extraction and qScript microRNA cDNA synthesis kit (Quanta Biosciences), or RNA was synthesized with a random hexamer using Maxima Reverse Transcriptase so that RNA was isolated from materials obtained by the ASO pull-down. The cDNA was evaluated for expression through qPCR analysis with SYBR (Kapa Biosystems) using Bio-Rad iCycler. For normalization of the ASO pull-down results, relative levels of U6 snRNA or GAPDH mRNA in each sample were quantified.

### EXAMPLE 7

### Immunoblot Assay

Muscle tissue and isolated muscle cells were homogenized in a lysis buffer (50 mM Tris-Cl, pH 7.4, 150 mM NaCl, 0.5% Triton X-100, 1 mM EDTA, 1 mM MgCl₂) which contains a protease and a phosphatase inhibitor. The lysate was centrifuged at 15,000×g for 20 minutes at 4° C., and the resulting supernatant was subjected to SDS-PAGE followed by immunoblot assay. Antibodies used for immunoblotting include ACTB (β-actin, Abcam), ACTN1 (Santa Cruz Biotechnology), AKT (Santa Cruz Biotechnology), mTOR (Cell Signaling Technology), S6K (Cell Signaling Technology), 4EBP (Cell Signaling Technology), FOXO3a (Cell Signaling Technology), MuRF1 (Santa Cruz Biotechnology), Atrogin-1 (Thermo scientific, ECM), and eIF3f (Novus). For GAPDH, an antibody developed in-house was used. ACTB, ACTN1, or GAPDH was used for normalization.

### EXAMPLE 8

### Morphometric Cytology Analysis

For immunostaining, differentiated C2C12 myotube cells were fixed with 4% paraformaldehyde and incubated with 0.3% Triton X-100 to improve permeability. The fixed sample was blocked with PBS containing 3% FBS, treated with anti-MyHC (Santa Cruz Biotechnology), washed with PBS, and reacted with AlexaFluor 488 (Invitrogen) secondary antibody. For Eosin staining, the differentiated C2C12 myotube cells were fixed in cold methanol for 15 minutes at −20° C. and stained with Eosin Y (Thermo Scientific) for 15 minutes.

Samples were washed three times with distilled water and images thereof were analyzed using a Nikon Eclipse Ti-U microscope. In order to analyze diameters of the myotube cells, four images were randomly selected. Diameters of the myotube cells in the selected images were calculated using a microscope imaging software (NIS-Elements Basic Research, Nikon). Genomic DNA was isolated using a specific genomic DNA kit (NANOHELIX), and a protein concentration in the cell lysate was analyzed by BCA protein analysis reagent (Pierce) to measure a ratio of protein to genomic DNA.

For immunohistochemical analysis, mouse TA muscle tissue was fixed in 4% paraformaldehyde and infiltrated with 15% to 30% sucrose. Frozen mouse muscle sections having 10 _{i—}tm in thickness were made using a cryostat (Leica) and stained with DAPI and antibodies according to a standard protocol. Samples were blocked with 3% FBS in PBS containing 0.05% Tween-20, treated with anti-laminin (Sigma-Aldrich), washed with PBS, and reacted with AlexaFluor 546 (Invitrogen) secondary antibody. For measurement of cross section area, six images were randomly selected. Cross section areas of the images were calculated using NIH ImageJ software.

### EXAMPLE 9

### Statistical Analysis

Quantitative data were presented as mean ±standard deviation unless otherwise specified. Difference in means was evaluated using Student's unpaired t test, and P<0.05 was analyzed to be statistically significant.

### EXPERIMENTAL EXAMPLE 1

### Expression of miRNAs Located in Dlk1-Dio3 Cluster in Muscle with Age

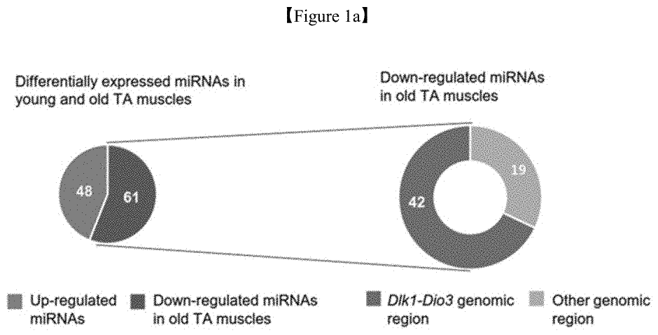

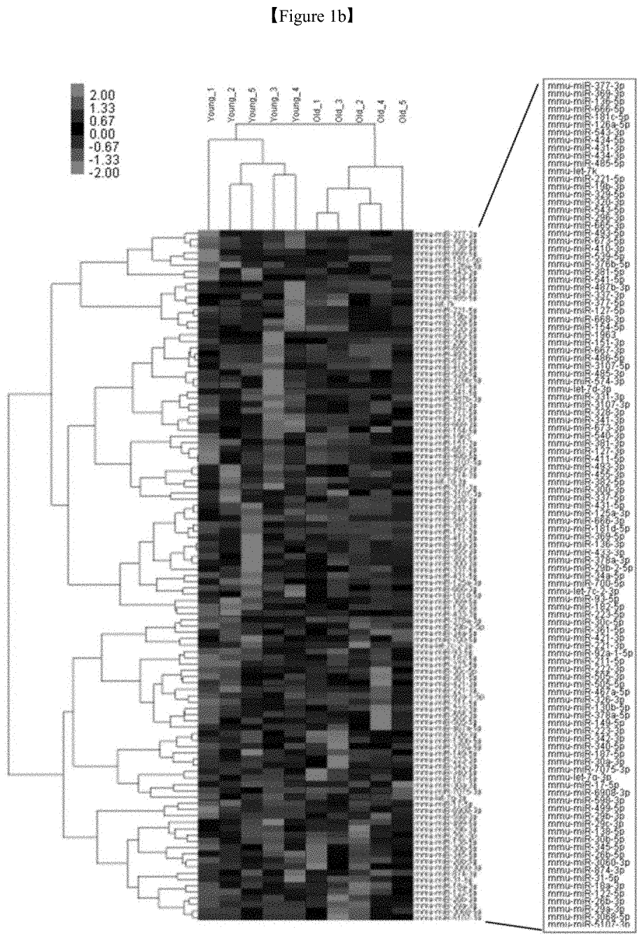

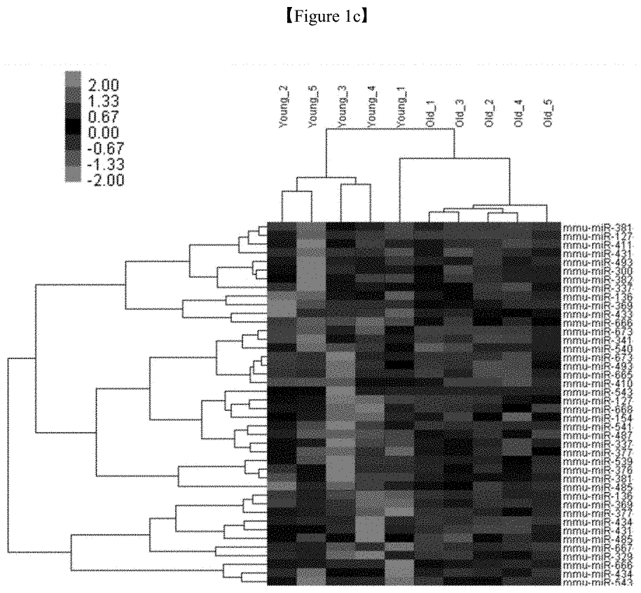

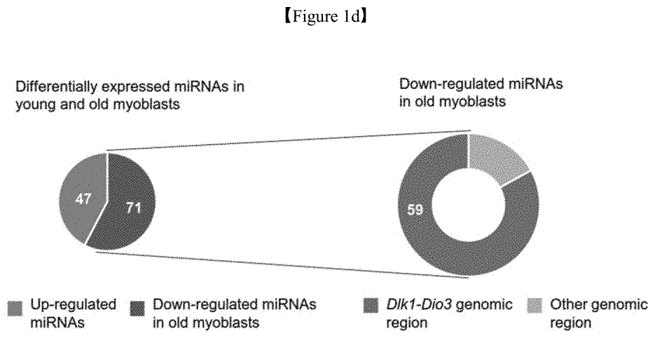

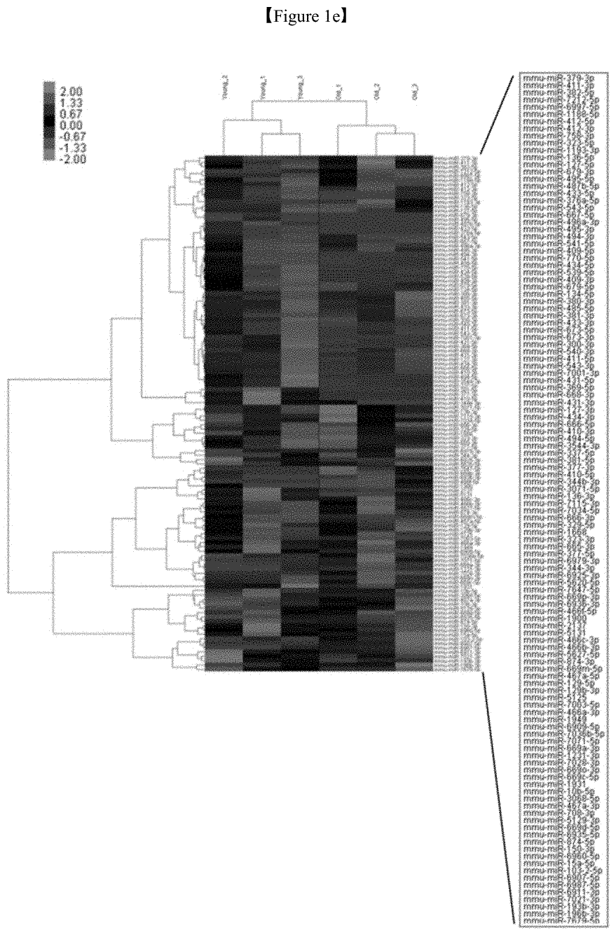

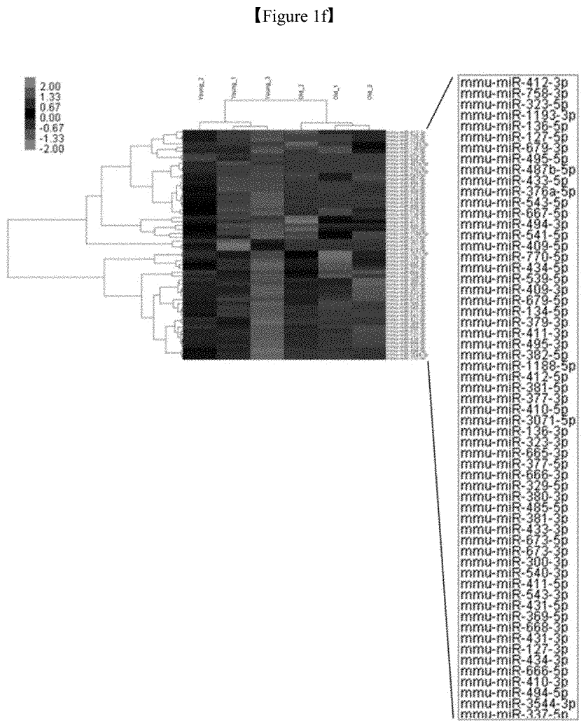

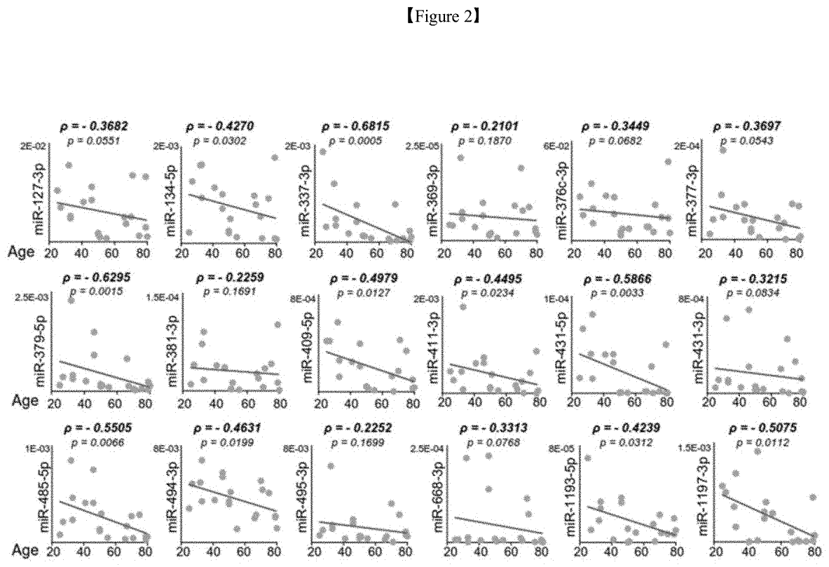

Expression patterns, with age, of miRNAs located in the Dlk1-Dio3 cluster in human skeletal muscle tissue were examined. Human Dlk1-Dio3 gene locus contains 99 mature miRNAs (54 pre-miRNAs) among which 87 are conserved between human and mouse genomes. Skeletal muscle tissue samples (n=20) were obtained from human participants aged from 25 to 80, and expression of 18 pre-miRNAs which had been randomly selected among the miRNAs present in Dlk1-Dio3 was analyzed by qRT-PCR. Results are shown in FIGS. 1a to 1d, and FIG. 2.

As shown in FIGS. la to ld, and FIG. 2, 10 pre-miRNAs showed significantly decreased expression with age (P<0.05), and the remaining 8 pre-miRNAs showed a tendency of decreased expression with age. Consistent decreased expression patterns, with age, of the miRNAs located in the Dlk1-Dio3 cluster in mouse and human muscles suggest an important role of these molecules in muscle aging.

### EXPERIMENTAL EXAMPLE 2

### Confirmation of Effects of miRNAs Located in Dlk1-Dio3 Cluster on Diameters of Myotube Cells

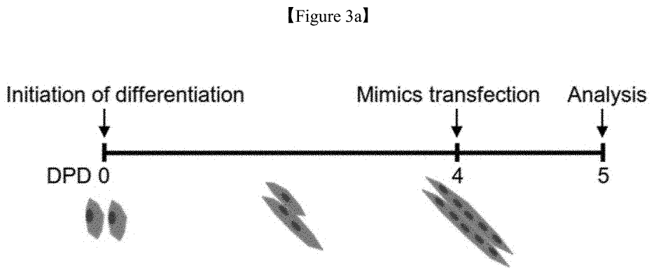

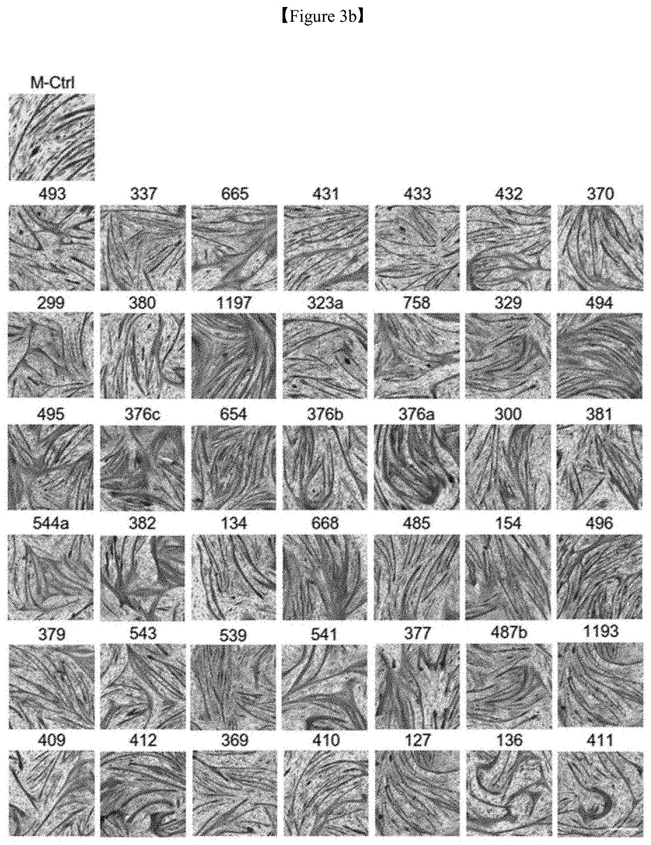

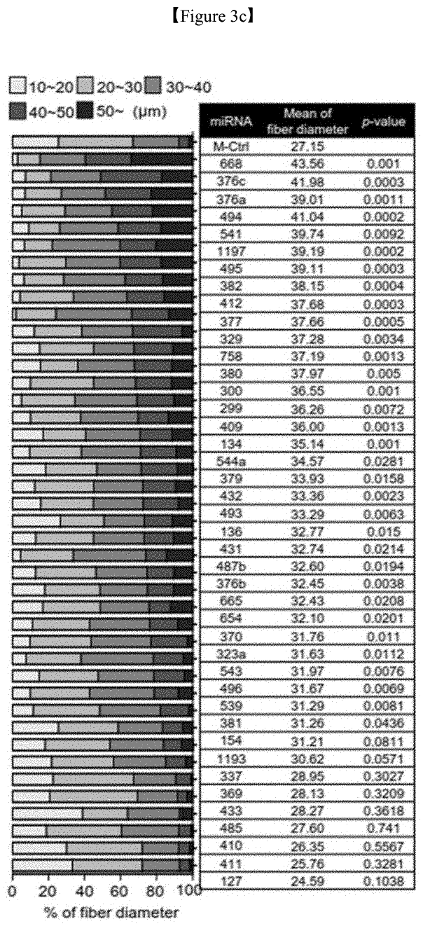

In order to examine whether miRNAs whose sequences were conserved between mouse and human, among miRNAs located at the Dlk1-Dio3 gene locus, are involved in muscular atrophy which was one of main phenotypes of aged muscles, miRNA mimics were over-expressed in C2C12 cells, which had been fully differentiated into myotube cells, so as to evaluate effects thereof on diameters of the myotube cells. A procedure for the above experiment is shown in FIG. 3a, and results of the above experiment are shown in FIGS. 3b and 3c.

As shown in FIGS. 3b and 3c, 36 of 42 pre-miRNAs tested induced a remarkably larger diameter than a control. From the results, a possibility that the miRNAs present in the Dlk1-Dio3 cluster contribute to skeletal muscular atrophy with aging can be confirmed, and it can be seen that administration of the miRNAs allows a skeletal muscle size to increase.

### EXPERIMENTAL EXAMPLE 3

### miRNAs Located in Dlk1-Dio3 Cluster which Regulate Expression of Atrogin-1 Protein

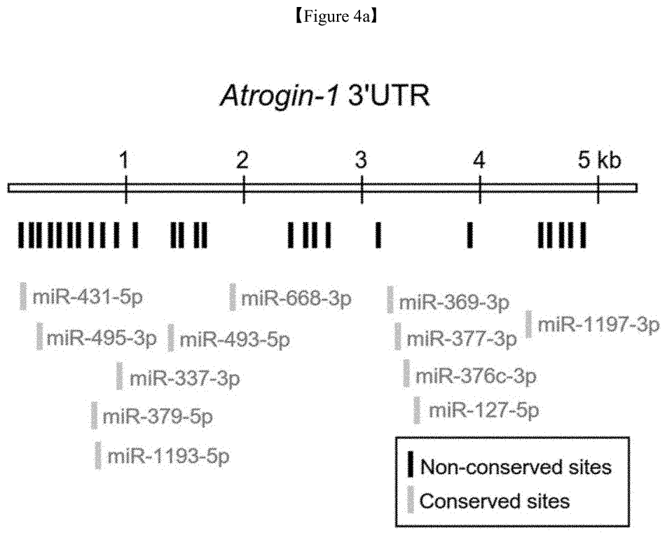

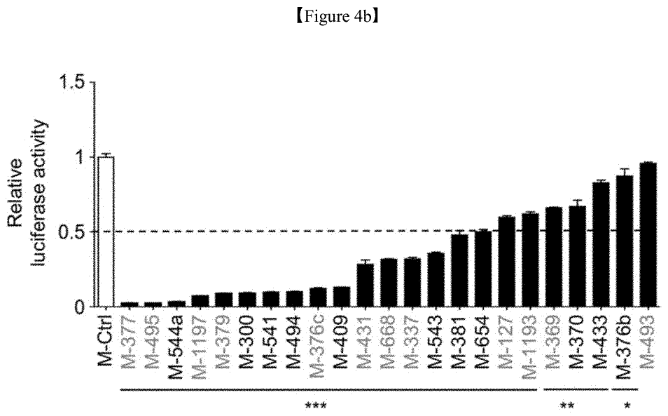

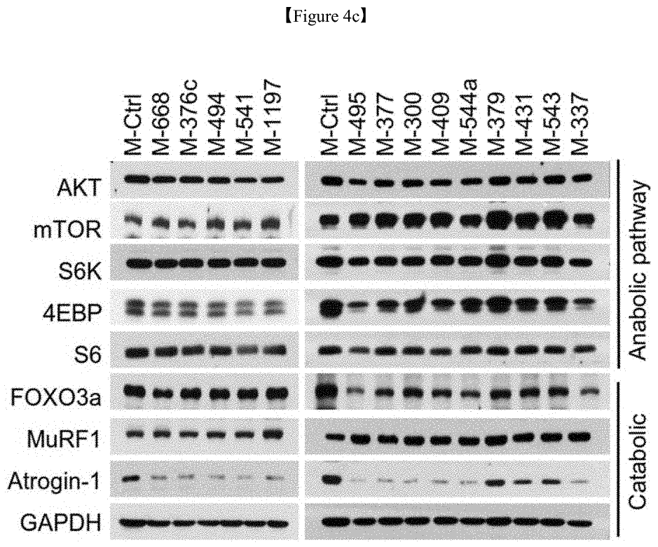

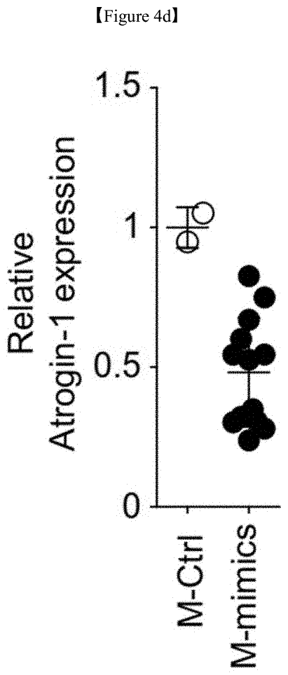

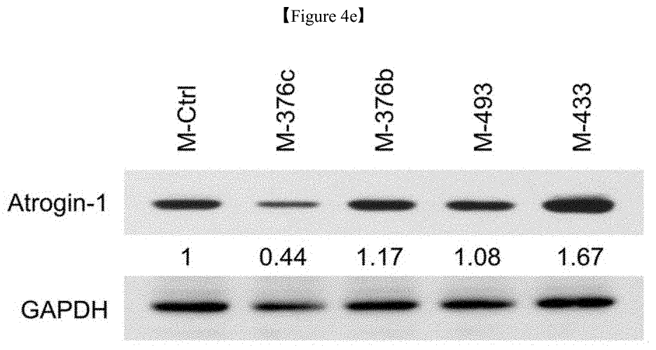

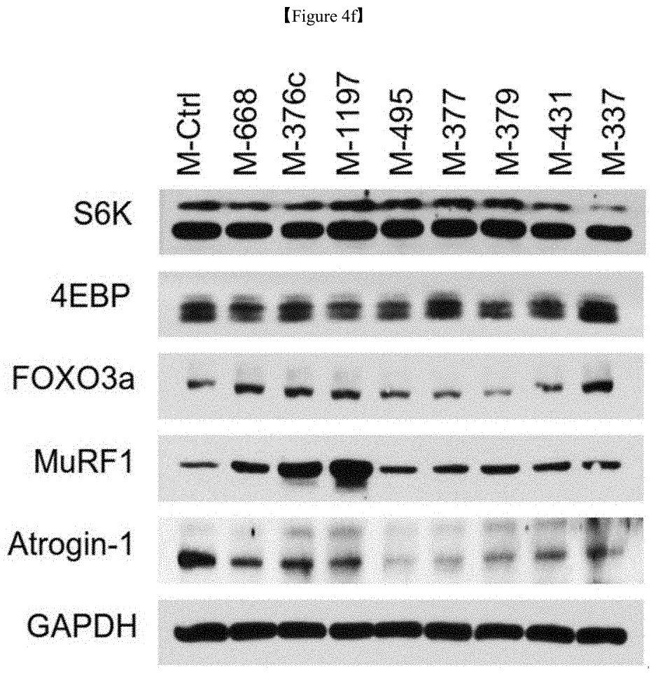

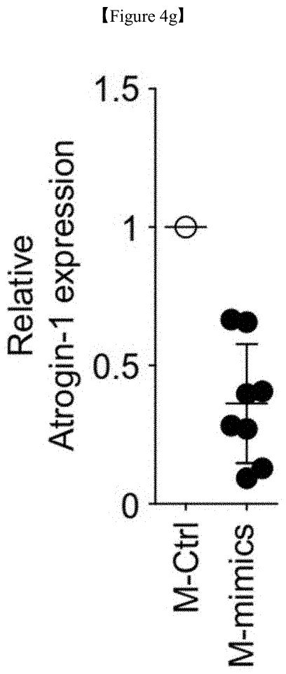

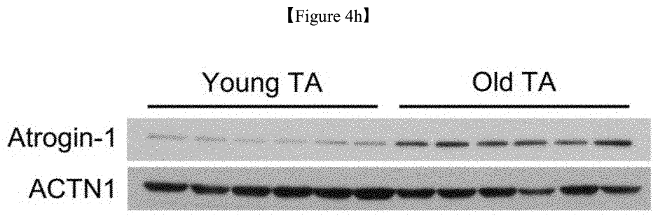

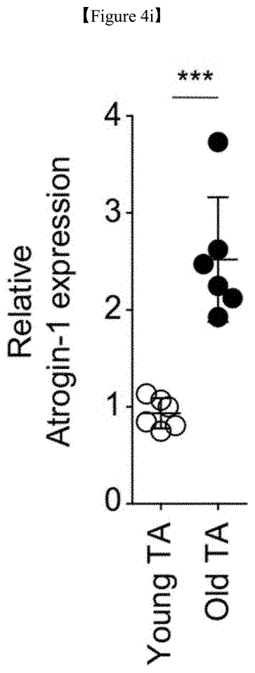

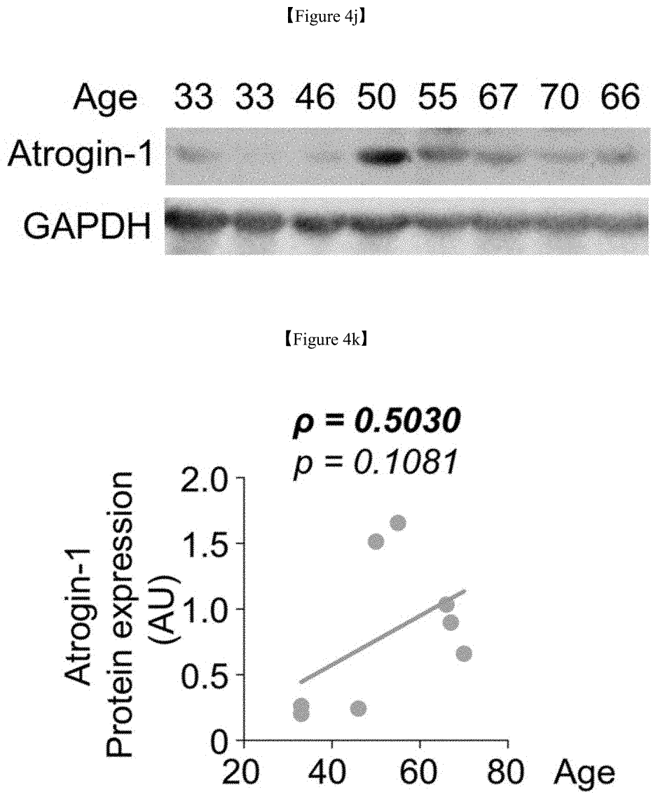

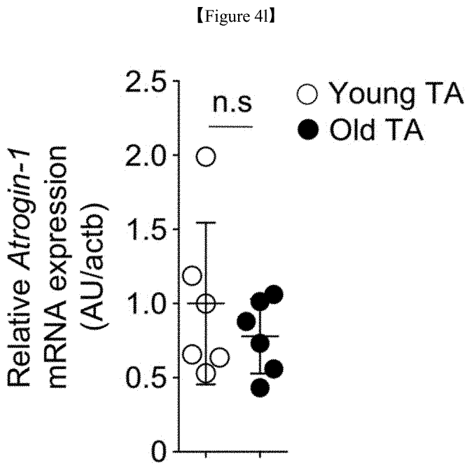

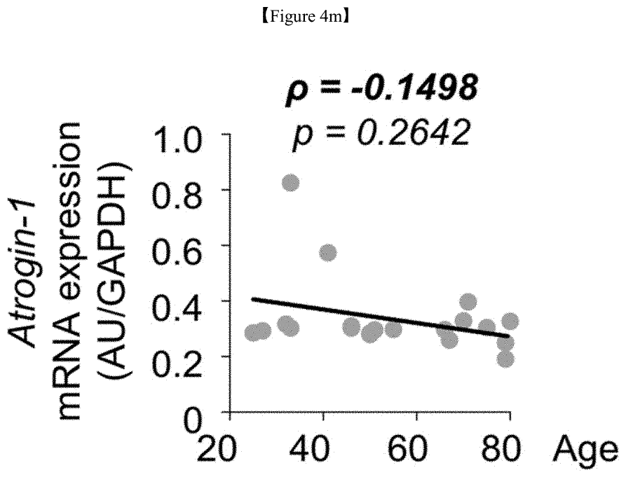

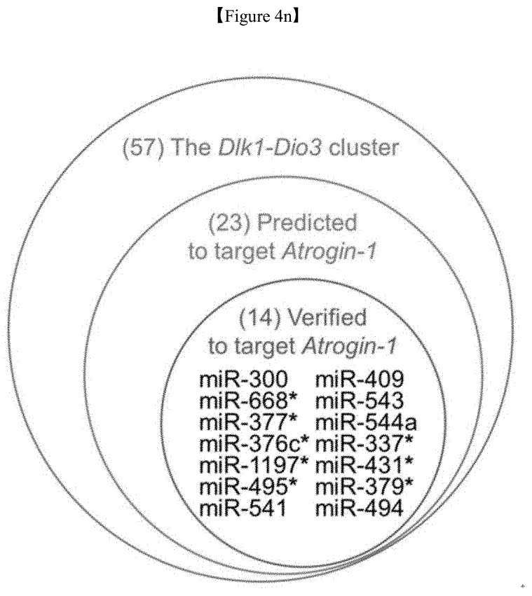

TargetScan algorithm (www.targetscan.org) was used to identify targets of miRNAs which mediate an anti-atrophic phenotype observed in mimic-transfected myotube cells. The results are shown in FIGS. 4a to 4n.

38 mature miRNAs originating from 23 pre-miRNAs were predicted to target 3′UTR of Atrogin-1 that encoded a muscle-specific E3 ligase (FIG. 4a, Table 7). Here, * in Table 7 below means a conserved position in human Atrogin-1 3′UTR.

TABLE 7

| pre-miRNA | mature | Number of binding | |
|---|---|---|---|
| (23) | miRNA (27) | sites (38) | Position (bp) |
| 127 | 127-5p | 3 | 3218-3224 |
| | | | 3622-3628* |
| | | | 3977-3983 |
| 300 | 300-3p | 1 | 5480-5486 |
| 337 | 337-3p | 1 | 1013-1019* |
| 369 | 369-3p | 2 | 3638-3644* |
| | | | 5305-5312 |
| 370 | 370-3p | 2 | 464-470 |
| 376b | 376b-3p | 1 | 518-524 |
| | 376b-5p | 1 | 480-486 |
| 376c | 376c-3p | 2 | 275-281 |
| | | | 3781-3787* |
| | 376c-5p | 1 | 480-486 |
| 377 | 377-3p | 1 | 3721-3728* |
| 379 | 379-5p | 1 | 539-545* |
| 381 | 381-3p | 1 | 5512-5518 |
| 409 | 409-5p | 1 | 4511-4517 |
| 431 | 431-5p | 1 | 253-259* |
| 433 | 433-3p | 1 | 787-793 |
| 493 | 493-5p | 1 | 1301-1307* |
| 494 | 494-3p | 2 | 601-607 |
| | | | 1129-1135 |
| 495 | 495-3p | 2 | 455-461* |
| | | | 5427-5433 |
| 541 | 541-5p | 1 | 5231-5237 |
| 543 | 543-3p | 1 | 1412-1418 |
| 544 | 544-3p | 3 | 206-212 |
| | | | 1386-1392 |
| | | | 2943-2949 |
| | 544-5p | 3 | 341-347 |
| | | | 1489-1495 |
| | | | 1714-1720 |
| 654 | 654-3p | 1 | 3274-3280 |
| 668 | 668-3p | 1 | 2183-2190* |
| 1193 | 1193-3p | 1 | 1428-1434 |
| | 1193-5p | 1 | 539-545* |
| 1197 | 1197-3p | 1 | 5026-5032* |

A luciferase reporter assay was used to confirm whether these miRNAs actually target the Atrogin-1 3′UTR. As a result, 14 of the 23 pre-miRNAs remarkably decreased reporter activity to equal to or less than half (FIG. 4b), indicating that at least 14 pre-miRNAs in the Dlk1-Dio3 cluster are capable of directly binding to the Atrogin-1 3′UTR. In C2C12 muscle cells transfected with the pre-miRNAs, the expression of Atorgin-1 protein was specifically decreased while there was no change in that of main proteins, which are involved in muscle homeostasis, such as AKT, mTOR, S6K, 4EBP, FOXO3a, and MuRF1.

6 miRNAs (miR-381, 654, 127, 1193, 369, and 370) decreased reporter activity by 33% to 50% as compared with a control, and 3 miRNAs (miR-433, 376b, and 493) had no effects on the expression of Atrogin-1 (FIG. 4e). Also in human skeletal muscle myoblasts (HSMMs), the conserved miRNAs resulted in suppressed expression of Atrogin-1 while having no effects on other main proteins (FIGS. 4f and 4g). In addition, as can be inferred from the fact that the miRNAs located in the Dlk1-Dio3 cluster exhibit remarkably decreased expression in aged muscle tissue, it can be seen that the expression of Atrogin-1 protein increases with age in both mice and humans as compared with young muscle tissue (FIGS. 4h to 4k).

However, there was no difference in the expression of Atrogin-1 gene, indicating that increase of the expression of Atrogin-1 protein with age is due to post-transcriptional regulation mediated by the miRNAs in the cluster (FIGS. 4l and 4m). These results show that a specific group of the miRNAs in the Dlk1-Dio3 cluster (FIG. 4n) controls expression of the Atrogin-1 protein and that decreased expression, with aging, of the miRNAs located in the Dlk1-Dio3 cluster causes increased expression of Atrogin-1, and thus leads to sarcopenia.

### EXPERIMENTAL EXAMPLE 4

### Confirmation of Effects of Over-Expressed miR-376c-3p in Muscle on Aging-Induced Sarcopenia

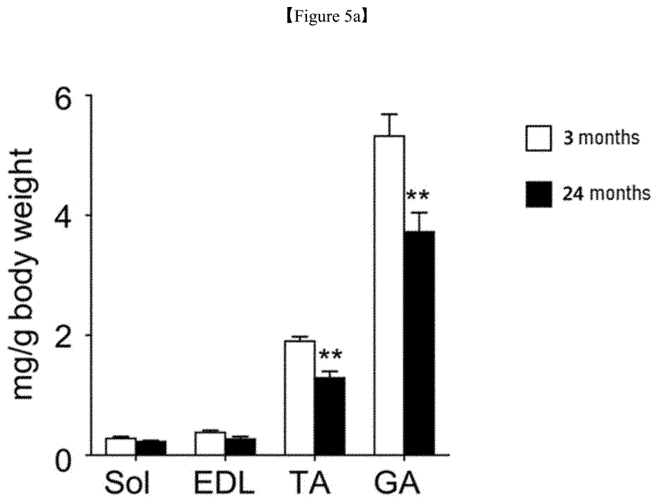

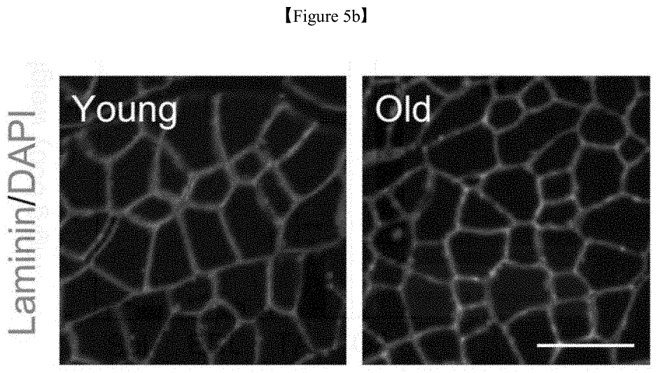

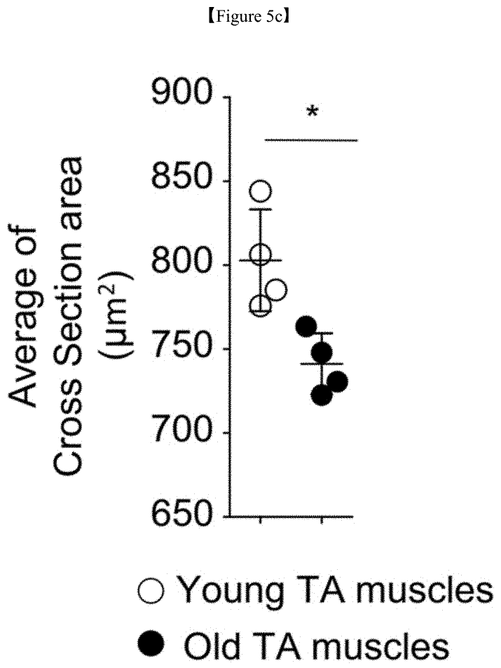

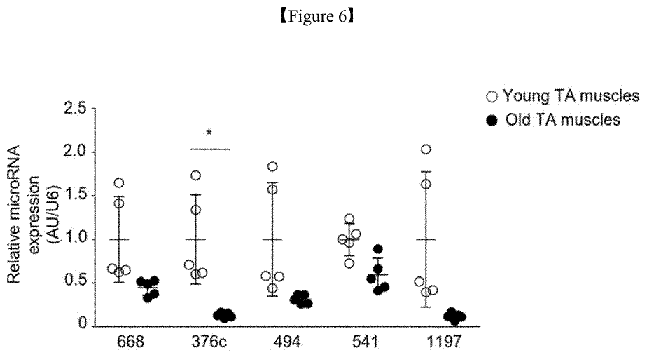

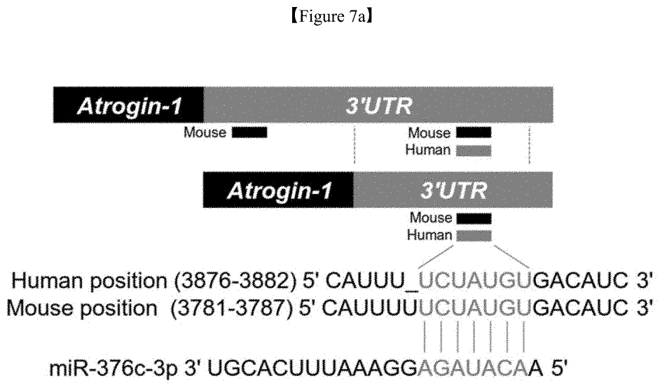

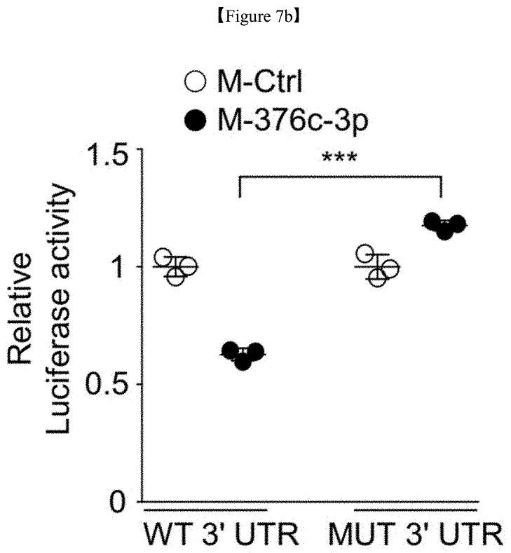

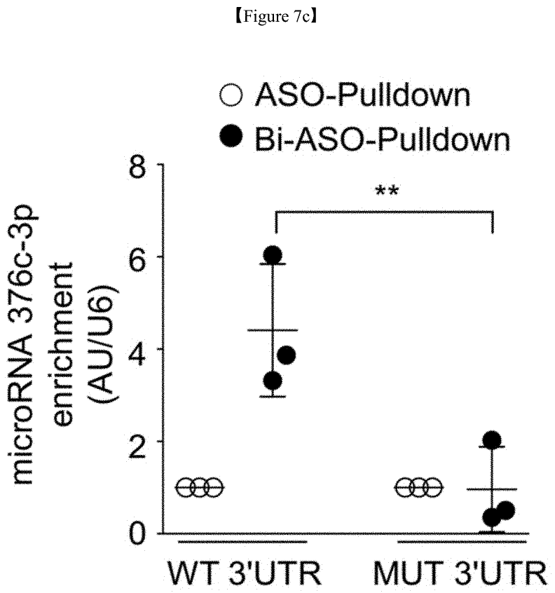

Muscle tissue of old mice at 24 months old showed a phenotype of remarkably decreased muscle mass and small cross section area as compared with muscle tissue of young mice at 3 months old (FIGS. 5a to 5c). In order to perform in vivo examination of therapeutic potential of the miRNAs in the Dlk1-Dio3 cluster, one of the most effective miRNAs that induced myotubes to have a large diameter was selected. Among top five miRNAs (FIG. 3c) which had greatly increased diameters of the myotubes, expression of miR-376c-3p was most definitely inhibited in aged TA muscles. Thus, this miRNA were finally selected (FIG. 6). In order to further examine specific interactions between the miR-376c-3p and the Atrogin-1 3′UTR, a reporter assay was performed using a luciferase Atrogin-1 3′UTR construct and a miR-376c-3p mimic. The miR-376c-3p mimic (miR-376c-3p) decreased luciferase activity, and such decreased activity disappeared in a case where a miR-376c-3p binding site on the 3′UTR was removed (FIGS. 7a and 7b).

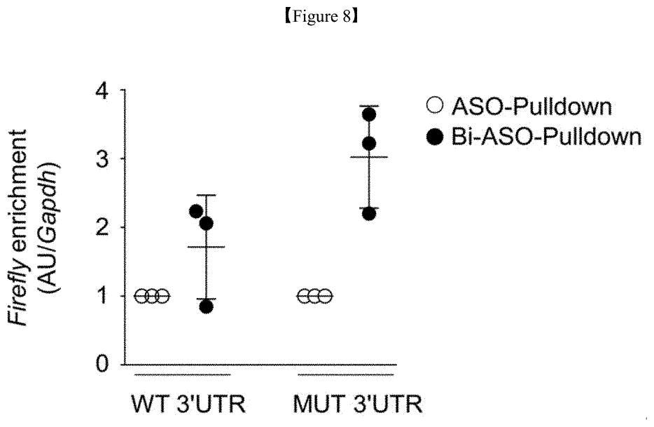

Pull-down experiments performed using biotinylated Atrogin-1 antisense oligomers (Bi-ASOs) demonstrated direct binding between miR-376c-3p and Atrogin-1 3′UTR which are intrinsically present (FIGS. 7c and 8). Specifically, C2C12 cells were transfected with a luciferase reporter having Atrogin-1 3′UTR that contains a wild-type or deletion-mutated miR-376c-3p binding site. 48 hours after transfection, Luciferase 2 mRNA was extracted using streptavidin beads and ASO (which is used for analysis in the presence or absence of biotin) in RT-qPCR analysis, to detect luciferase 2 mRNA enrichment.

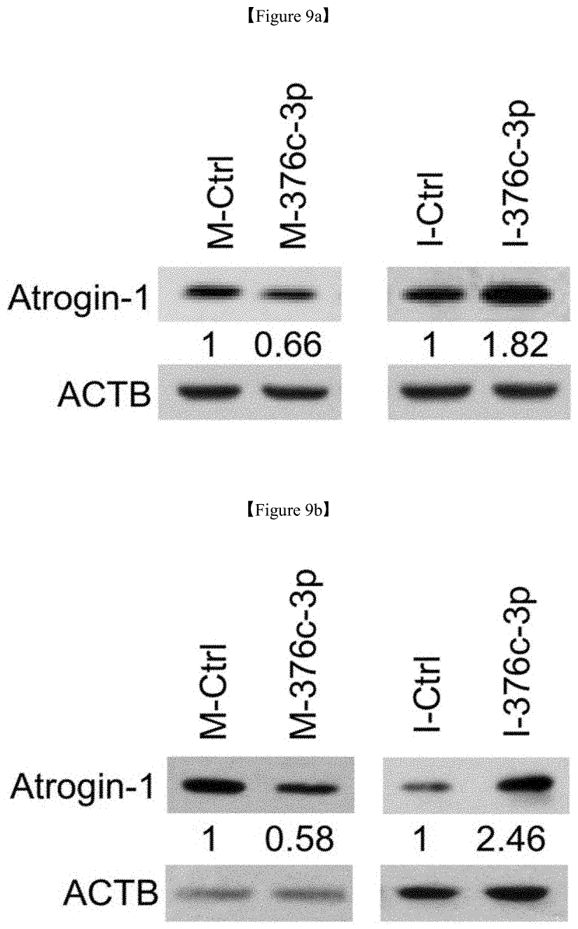

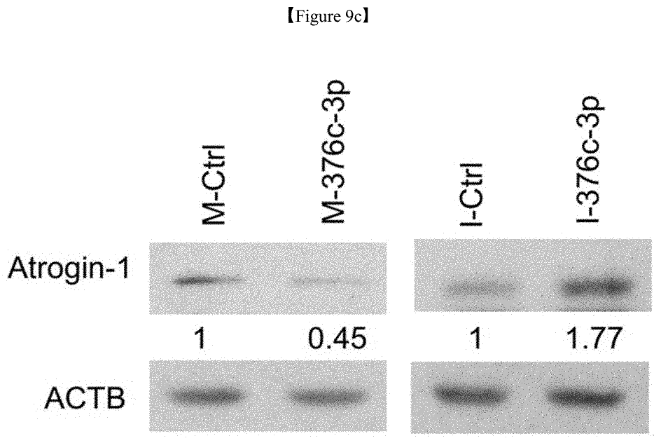

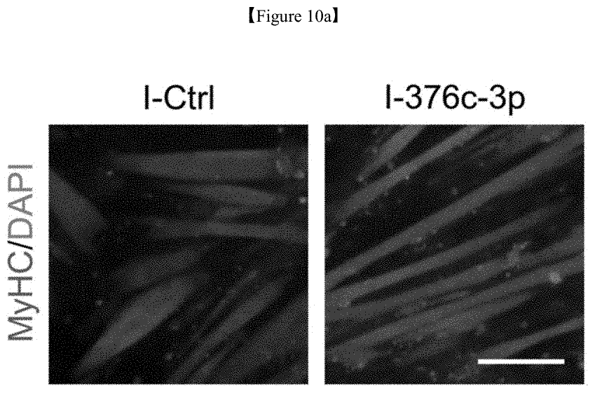

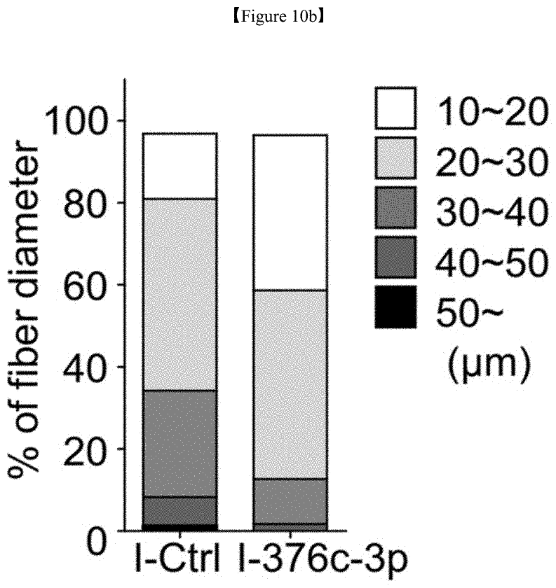

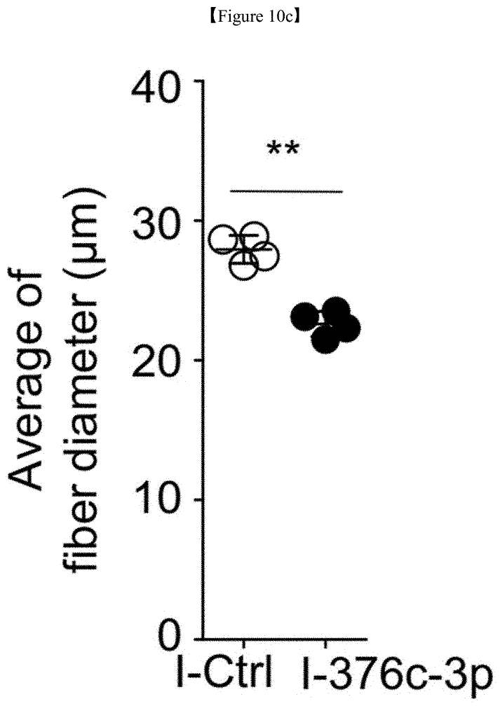

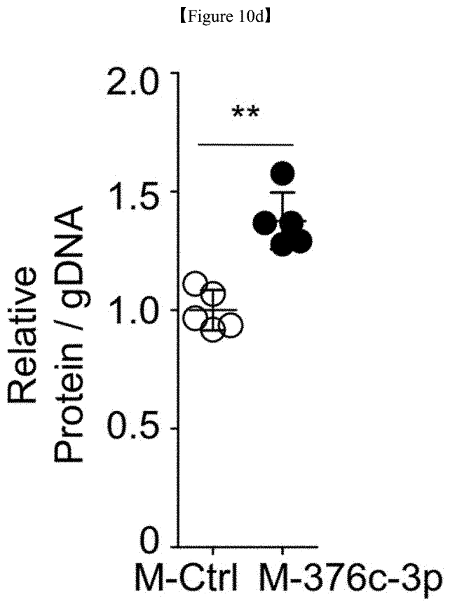

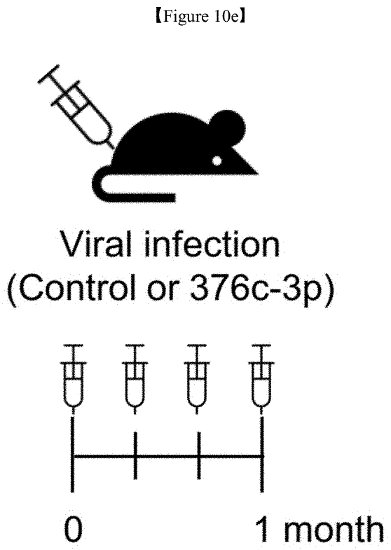

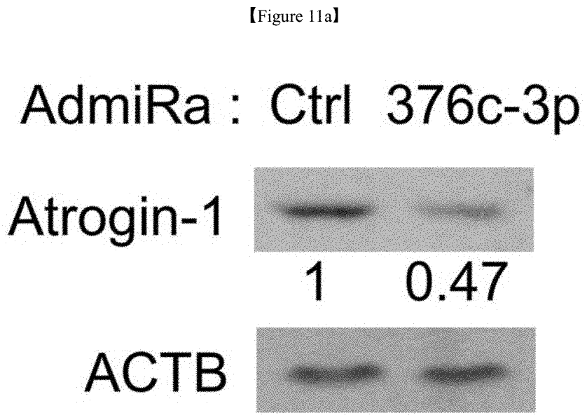

miR-376c-3p transfection decreased expression of Atrogin-1 in all of primary myoblasts, C2C12 cells, and HSMIMs. On the contrary, inhibitor (I)-mR-376c-3p increased expression of Atrogin-1 (FIGS. 9a to 9c). Myotubes transfected with miR-376c-3p also exhibited decreased fiber thickness (FIGS. 10a to 10c), and HSMIMs transfected with miR-376c-3p exhibited significantly increased total intracellular protein content (FIG. 10d).

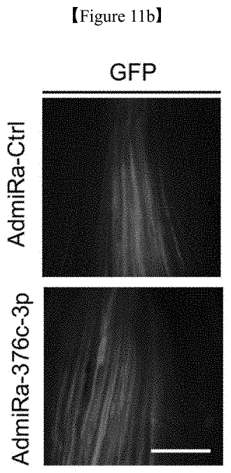

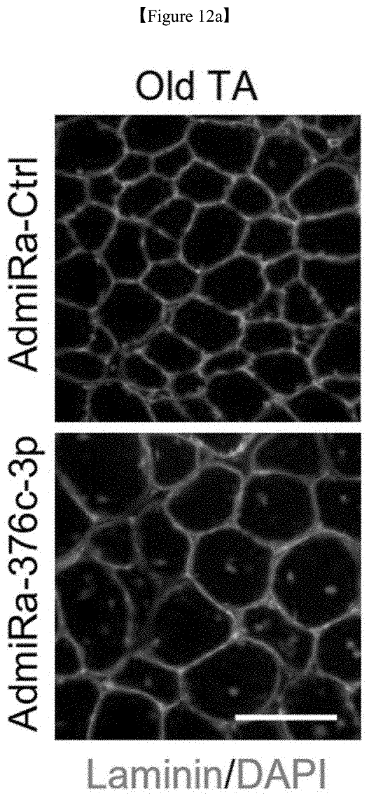

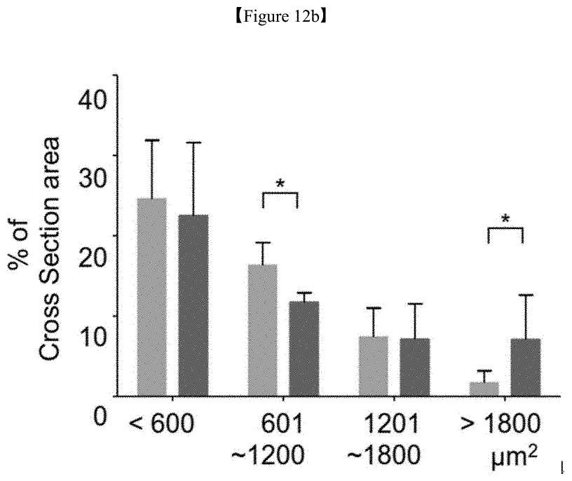

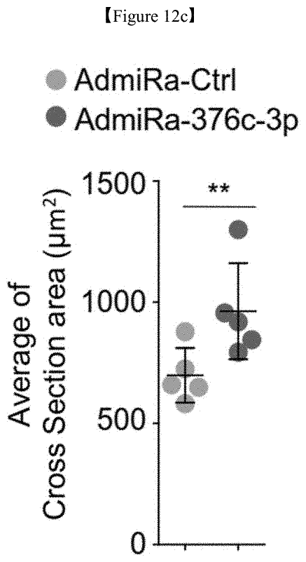

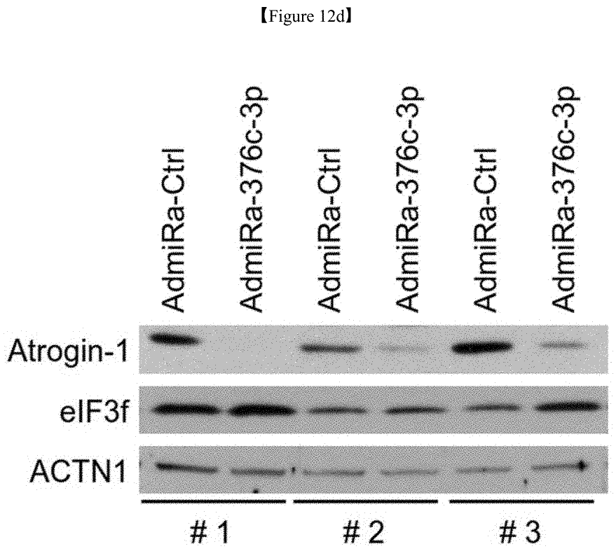

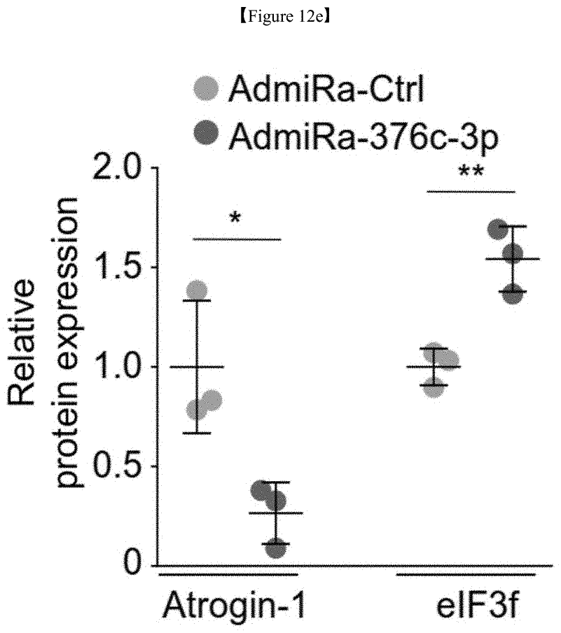

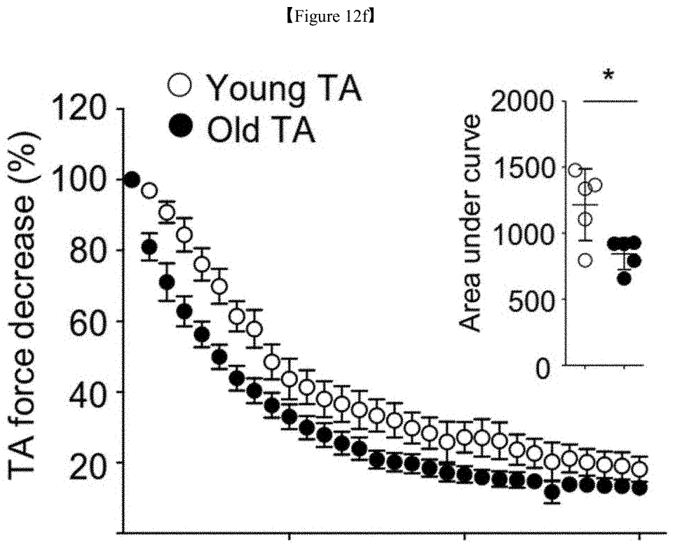

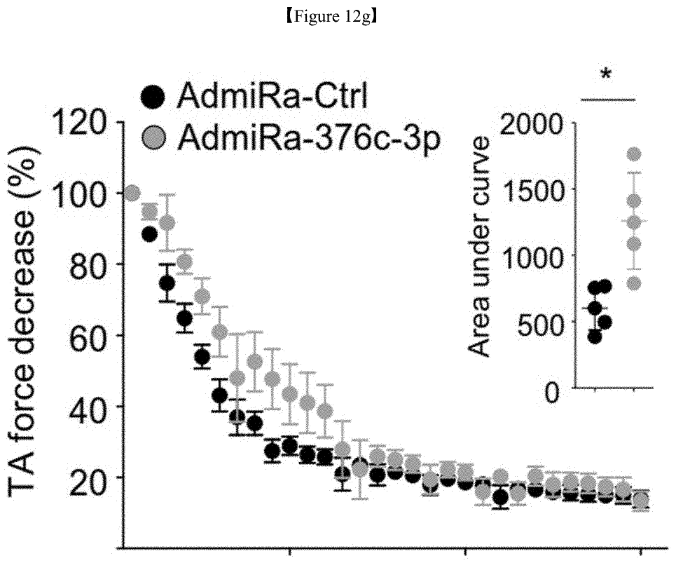

Finally, in order to confirm whether miR-376c-3p improved decrease of muscle in old mice, miR-376c-3p (AdmiRa-376c-3p) or control adenovirus (AdmiRa-Ctrl) was administered to TA muscles of 23-month-old mice for 1 month, and cross-sections were examined by immunohistochemical analysis (FIGS. 10e, 11a, and 11b). Here, six mice were used as mice for each experimental group. TA muscles over-expressing miR-376c-3p showed remarkably larger muscle fibers than the control (FIGS. 12a to 12c). Muscles infected with

AdmiRa-376c-3p exhibited increased expression level of eIF3f, which is known as a target of Atrogin-1 E3 ligase, in contrast to decreased expression of Atrogin-1 (FIGS. 12d and 12e). In addition, results of improving muscle fatigue were obtained, which showed a possibility of improving even functions of aged muscles (FIGS. 12f and 12g). These results show that miR-376c-3p can be an effective target for eradicating muscle aging.

### EXPERIMENTAL EXAMPLE 5

### Confirmation of Effects of miR-376c-3p on Muscular Atrophy Caused By Glucocorticoid and Cachexia

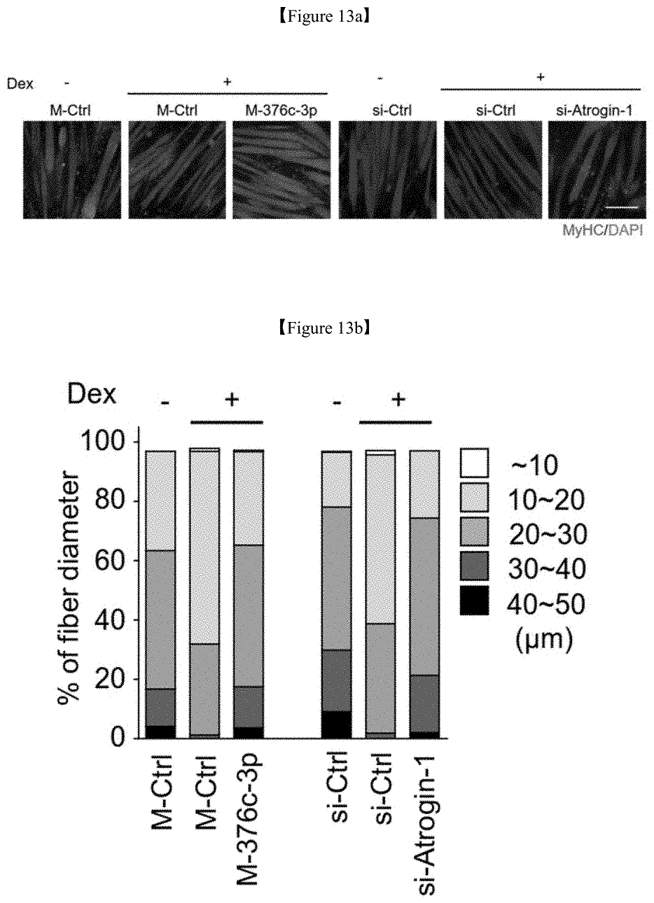

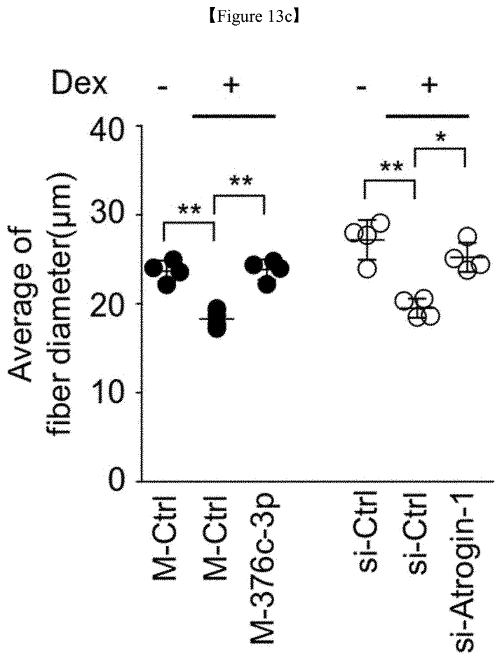

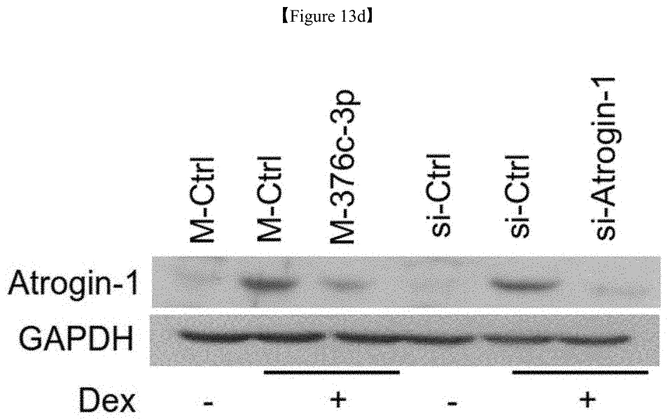

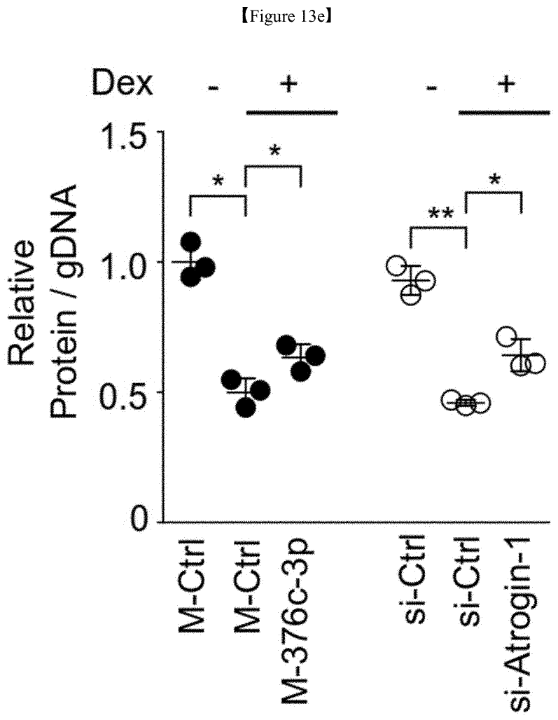

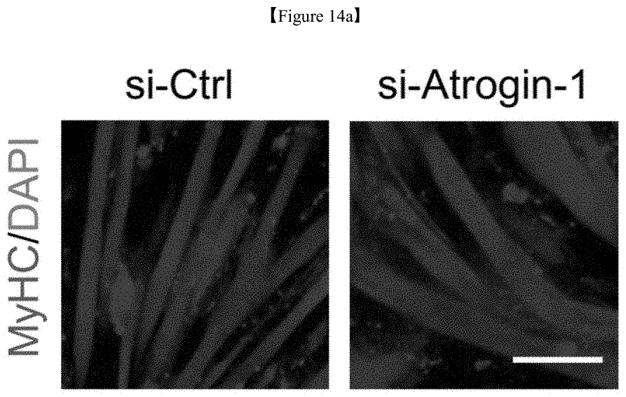

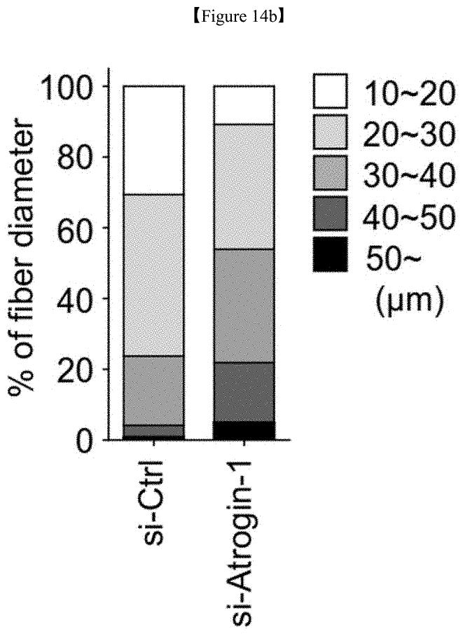

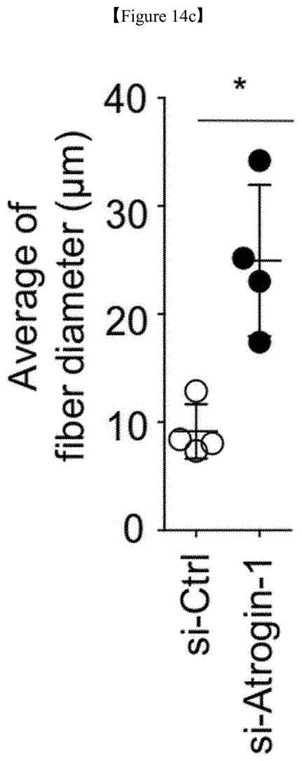

Atrogin-1 leads to atrophy in not only aged muscles in vivo but also muscles in vivo in which glucocorticoid is present or cancer has developed. Thus, it was confirmed that miR-376c-3p is capable of improving muscular atrophy caused by glucocorticoid. The results are shown in FIGS. 13a to 13e, and FIGS. 14a to 14c.

As shown in FIGS. 13a to 13e, and 14a to 14c, miR-376c-3p prevented muscular atrophy in myotubes which is induced by dexamethasone, and thus caused the myotubes to show a similar diameter to a control without dexamethasone treatment even in a case where muscular atrophy is caused by dexamethasone (FIGS. 13a to 13c). miR-376c-3p also decreased the expression of Atrogin-1, which had increased due to dex treatment, to a control level (FIG. 13d). In addition, miR-376c-3p returned the amount of intramuscular proteins, which had decreased due to dexamethasone treatment, to normal (FIG. 13e). Knock-down experiments of Atrogin-1 using siRNA confirmed that morphological deterioration of muscles is prevented by suppressed expression of Atrogin-1 protein in myotubes treated with dexamethasone (FIGS. 13a to 13e, and FIGS. 14a to 14c).

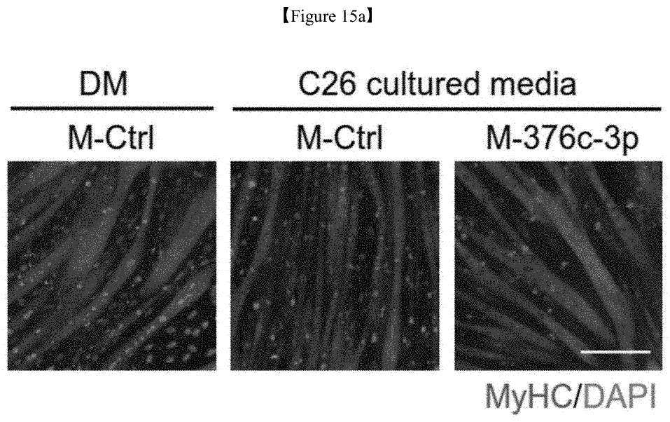

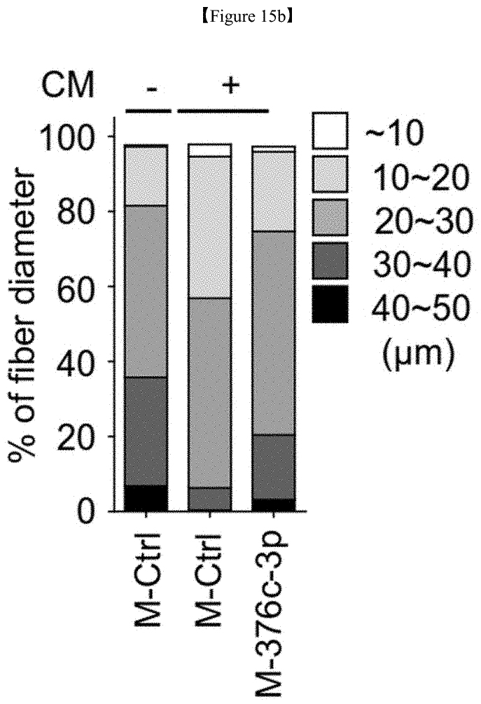

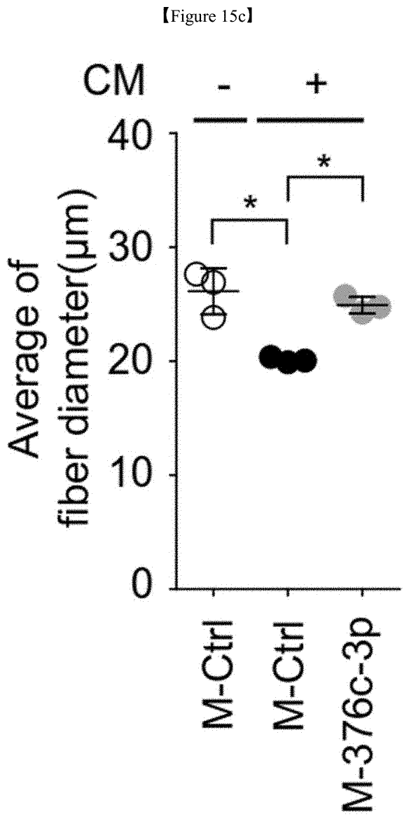

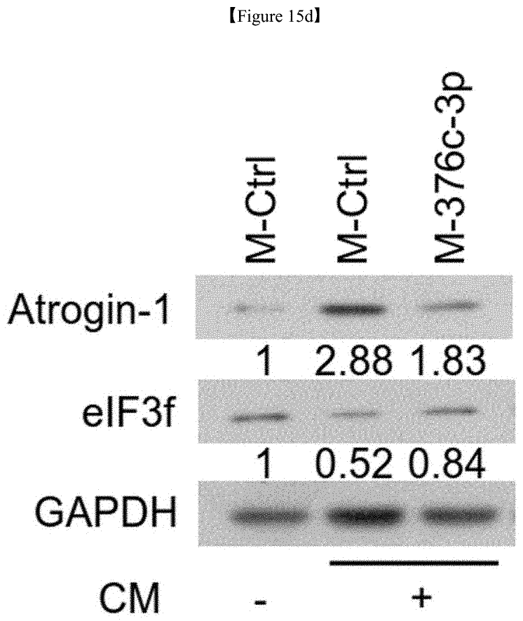

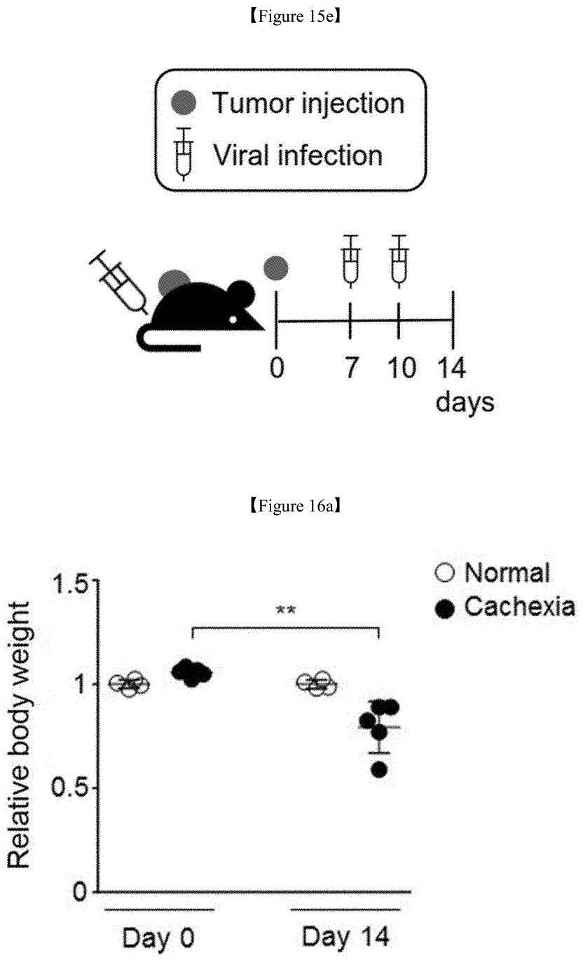

In addition, examination was performed on whether miR-376c-3p improved tumor-induced muscular atrophy. Atrogin-1 is an important marker for acute muscular atrophy and has been reported to be over-expressed in a case where cachexia develops. C2C12 myotubes were treated with a medium in which colon carcinoma cell line C26 had been cultured. As a result, it was possible to confirm that the myotubes become remarkably thinner. It was confirmed that transfection using miR-376c-3p restored muscular atrophy in the myotubes, which had been induced by the culture medium for C26, to a degree similar to that in myotubes of a control (FIGS. 15a to 15c). Under these conditions, the expression of Atrogin-1 decreased, and in contrast, the expression of eIF3f, which is known as a target of Atrogin-1, increased. Thus, it was confirmed that miR-376c-3p is capable of alleviating muscular atrophy in vitro (FIG. 15d).

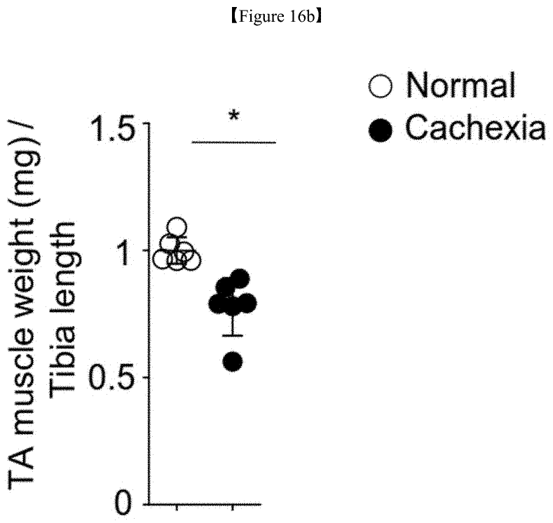

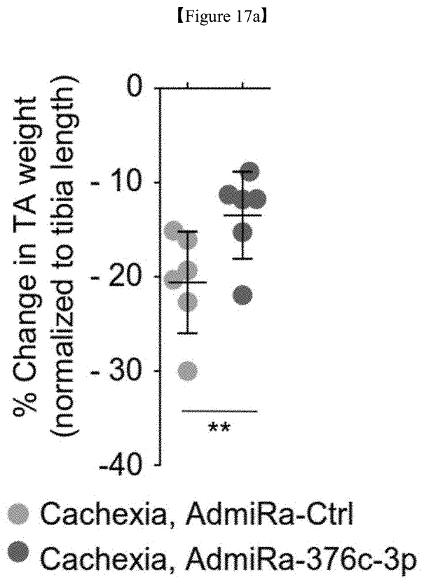

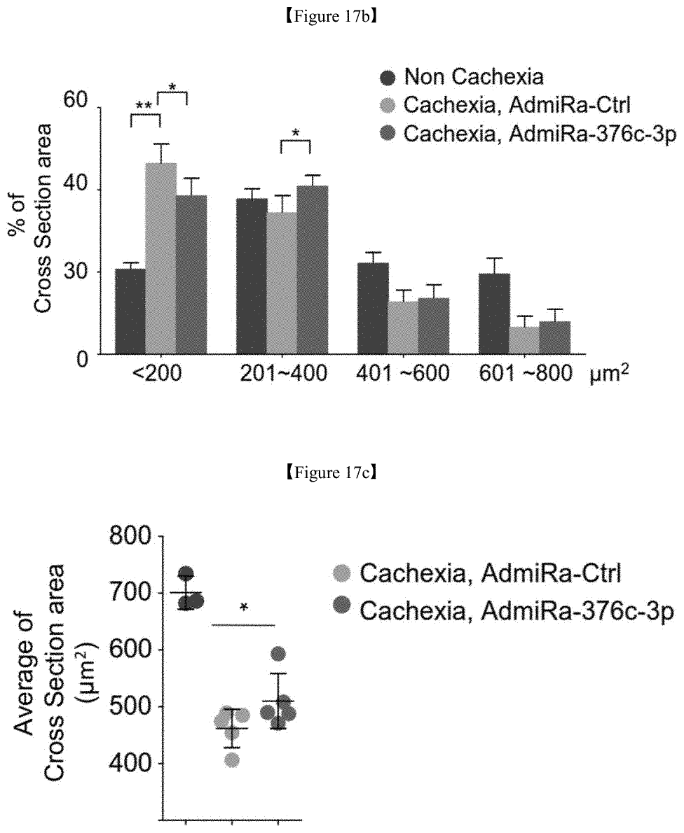

In addition, in order to confirm therapeutic potential of miR-376c-3p in a C26 tumor-induced cachexia mouse model, AdmiRa-Control or AdmiRa-376c-3p was injected into TA muscles on days 7 and 10 after inoculation of mice with C26 tumor, and observation was made on states of the muscles (FIG. 15e). It was confirmed that tumor-bearing mice exhibited a slightly lower body weight but significantly lower muscle mass than non-tumor mice (FIGS. 16a and 16b). It was observed that the TA muscles infected with AdmiRa-376c-3p showed a 13% decrease, whereas the contralateral TA muscles infected with AdmiRa-Control showed a 21% decrease (FIG. 17a). The TA muscles infected with AdmiRa-376c-3p were accompanied by an 8.5% increase in cross section area (FIGS. 17b and 17c). The experimental results show that over-expression of miR-376c-3p is capable of effectively suppressing muscular atrophy seen in tumor-induced cachexia.

1. A method of treating a muscular disease, comprising:
administering to a subject a pharmaceutical composition comprising a miRNA located in Dlk1-Dio3 cluster or a variant thereof.

2. The method according to claim 1, wherein the miRNA comprises at least one selected from the group consisting of miRNA-668, miRNA-376c, miRNA-494, miRNA-541, miRNA-377, miRNA-1197, miRNA-495, miRNA-300, miRNA-409, miRNA-544a, miRNA-379, miRNA-431, miRNA-543, and miRNA-337.

3. The method according to claim 1, wherein the miRNA decreases an expression level of Atrogin-1/MAFbx protein.

4. The method according to claim 1, wherein the miRNA directly interacts with 3′-untranslated region (3′-UTR) of a polynucleotide encoding Atrogin-1/MAFbx protein, and suppresses expression of Atrogin-1/MAFbx.

5. The method according to claim 1, wherein the muscular disease comprises at least one selected from the group consisting of sarcopenia, muscular atrophy, muscle dystrophy, and acardiotrophia.

6. A method of treating a muscular disease, comprising:
administering to a subject a pharmaceutical composition comprising a vector loaded with a nucleotide encoding a miRNA located in Dlk1-Dio3 cluster or a variant thereof.

7. The method according to claim 6, wherein the miRNA comprises at least one selected from the group consisting of miRNA-668, miRNA-376c, miRNA-494, miRNA-541, miRNA-377, miRNA-1197, miRNA-495, miRNA-300, miRNA-409, miRNA-544a, miRNA-379, miRNA-431, miRNA-543, and miRNA-337.

8. The method according to claim 6, wherein the vector comprises at least one selected from the group consisting of a plasmid vector, a cosmid vector, a virus, and analogs thereof.

9. The method according to claim 8, wherein the virus is adenovirus or adeno-associated virus.

10. A method of treating cachexia, comprising:
administering to a subject a pharmaceutical composition comprising a miRNA located in Dlk1-Dio3 cluster or a variant thereof.

11. The method according to claim 10, wherein the miRNA comprises at least one selected from the group consisting of miRNA-668, miRNA-376c, miRNA-494, miRNA-541, miRNA-377, miRNA-1197, miRNA-495, miRNA-300, miRNA-409, miRNA-544a, miRNA-379, miRNA-431, miRNA-543, and miRNA-337.

12. The pharmaceutical composition for preventing or A method of treating cachexia, comprising as an active ingredient:
administering to a subject a pharmaceutical composition comprising a vector loaded with a nucleotide encoding a miRNA located in Dlk1-Dio3 cluster or a variant thereof.

13. The method according to claim 12, wherein the miRNA comprises at least one selected from the group consisting of miRNA-668, miRNA-376c, miRNA-494, miRNA-541, miRNA-377, miRNA-1197, miRNA-495, miRNA-300, miRNA-409, miRNA-544a, miRNA-379, miRNA-431, miRNA-543, and miRNA-337.

14. The method according to claim 12, wherein the vector comprises at least one selected from the group consisting of a plasmid vector, a cosmid vector, a virus, and analogs thereof.

15. The method according to claim 14, wherein the virus is adenovirus or adeno-associated virus.

16-21. (canceled)
